(19) United States
(12) Patent Application Publication
(10) Pub. No.: US 2012/0107886 A1
(43) Pub. Date: May 3, 2012

(54) Lignin Oxidation and Products Thereof

(75) Inventors: Kim Albizati, San Diego, CA (US); Cara Tracewell, Solana Beach, CA (US)

(73) Assignee: Strategic Enzyme Applications, Inc., San Diego, CA (US)

(21) Appl. No.: 13/375,193

(22) PCT Filed: Jun. 1, 2010
(86) PCT No.: PCT/US10/36946

§ 371 Date: Jan. 9, 2012

Related U.S. Application Data
(60) Provisional application No. 61/183,017, filed on Jun. 1, 2009.
(60) Provisional application No. 61/287,140, filed on Dec. 16, 2009.

(51) Int. Cl. C07C 51/285; C12P 7/42; C07C 51/21

(52) U.S. Cl. 435/146; 562/407; 562/523

(57) ABSTRACT

Provided herein is a method of oxidizing lignin. Further disclosed herein are aromatic and non-aromatic compounds obtained from oxidized lignin.

### CROSS-REFERENCE

This application claims the benefit of U.S. Provisional Application No. 61/183,017, filed Jun. 1, 2009 and U.S. Provisional Application No. 61/287,140, filed Dec. 16, 2009, all of which are incorporated herein by reference.

### BACKGROUND OF THE INVENTION

Biomass means biological material that is used for industrial production (e.g., to generate chemical compounds). Sources of biomass includes, but is not limited to, trees, shrubs, grasses, wheat, wheat straw, sugar cane bagasse, corn, corn husks, corn kernel including fiber from kernels, products and by-products from milling of grains. Lignin comprises about 15% to about 30% the weight of dry wood.

### SUMMARY OF THE INVENTION

Described herein are compositions and methods for converting lignin (e.g., purified or native lignin) into small molecule products having a single phenyl moiety. The single phenyl moiety product is then optionally converted via at least one oxidation step into further desired products, including 2-methoxyphenol, catechol, phenol, muconic acid, adipic acid, butadiene, acrylic acid, methanol, 2-aminophenol, aniline, cyclohexyl carboxylic acid, caprolactam, ortho-phenylenediamine. Also described herein are the aforementioned desired products that are prepared from lignin (e.g., purified or native lignin).

Provided in certain embodiments herein is a method of obtaining vanillic acid from lignin, comprising: subjecting lignin to oxidative conditions sufficient to produce vanillic acid. In some embodiments, the lignin is isolated from biomass. In certain embodiments, the lignin is not isolated from biomass. In some embodiments, the oxidative conditions comprise contacting lignin with a liginase. In certain embodiments, the oxidative conditions comprise contacting lignin with an oxidase, a peroxidase, or both. In some embodiments, the oxidative conditions comprise contacting lignin with an oxidizing agent. In certain embodiments, the oxidative conditions comprise contacting lignin with molecular oxygen; hydrogen peroxide; or both. In some embodiments, the oxidative conditions comprise contacting lignin with (a) an oxidizing agent; and (b) a metal catalyst. In certain embodiments, the oxidative conditions comprise contacting lignin with (a) an oxidizing agent; and (b) a metal catalyst selected from a Group VII transition metal; a Group VA metal, or both. In specific embodiments, the oxidative conditions comprise contacting lignin with (a) an oxidizing agent; and (b) Co, Rh, Ir, MeReO₃, or combinations thereof. In some embodiments, the oxidative conditions comprise contacting lignin with (a) hydrogen peroxide; and (b) MeReO₃. In certain embodiments, provided herein is vanillic acid obtained according to any method described herein.

Also provided herein is a method of obtaining 2-methoxyphenol, comprising: (a) subjecting lignin to oxidative conditions sufficient to produce vanillic acid; and (b) subjecting vanillic acid to conditions sufficient to produce 2-methoxyphenol; and 2-methoxyphenol obtained thereby.

Provided in some embodiments herein is a method of obtaining catechol comprising: (a) subjecting lignin to oxidative conditions sufficient to produce vanillic acid; (b) subjecting vanillic acid to conditions sufficient to produce 2-methoxyphenol; and (c) subjecting 2-methoxyphenol to conditions sufficient to produce catechol; and catechol produced thereby.

Provided in certain embodiments herein is a method of obtaining phenol comprising: (a) subjecting lignin to oxidative conditions sufficient to produce vanillic acid; (b) subjecting vanillic acid to conditions sufficient to produce 2-methoxyphenol; (c) subjecting 2-methoxyphenol to conditions sufficient to produce catechol; and (d) subjecting 2-methoxyphenol to conditions sufficient to produce phenol; and phenol obtained thereby.

Also provided herein is a method of obtaining muconic acid: comprising: (a) subjecting lignin to oxidative conditions sufficient to produce vanillic acid; and (b) subjecting vanillic acid to conditions sufficient to produce muconic acid; and muconic acid obtained thereby.

Provided in some embodiments herein is a method of obtaining adipic acid: comprising: (a) subjecting lignin to oxidative conditions sufficient to produce vanillic acid; (b) subjecting vanillic acid to conditions sufficient to produce muconic acid; and (c) subjecting muconic acid to conditions sufficient to produce adipic acid; and adipic acid produced thereby.

In some embodiments, provided herein is a method of obtaining acrylic acid and butadiene from lignin, comprising: (a) subjecting lignin to oxidative conditions sufficient to produce vanillic acid, (b) subjecting vanillic acid to conditions sufficient to convert vanillic acid to catechol; (c) subjecting catechol to conditions sufficient to convert catechol to hexa-2,4-dienedioic acid; and (e) subjecting hexa-2,4-dienedioic acid to conditions sufficient to convert hexa-2,4-dienedioic acid to acrylic acid and butadiene; and acrylic acid or butadiene produced thereby.

In certain embodiments, provided herein is a method of obtaining an acrylate ester and butadiene from lignin, comprising: (a) subjecting lignin to oxidative conditions sufficient to produce vanillic acid, (b) subjecting vanillic acid to conditions sufficient to convert vanillic acid to catechol; (c) subjecting catechol to conditions sufficient to convert catechol to hexa-2,4-dienedioic acid; (e) esterifying hexa-2,4-dienedioic acid; and (e) subjecting the ester to conditions sufficient to convert the ester to an acrylate ester and butadiene; and acrylate or butadiene produced thereby.

In some embodiments, provided herein is a method of obtaining 2-aminophenol, comprising: (a) subjecting lignin to oxidative conditions sufficient to produce vanillic acid, (b) subjecting vanillic acid to conditions sufficient to convert vanillic acid to 4-amino-3-methoxybenzoic acid; and (c) subjecting 4-amino-3-methoxybenzoic acid to conditions sufficient to convert 4-amino-3-methoxybenzoic acid to 2-aminophenol; and 2-aminophenol obtained thereby.

Provided in certain embodiments herein is a method of obtaining aniline, comprising: (a) subjecting lignin to oxidative conditions sufficient to produce vanillic acid, (b) subjecting vanillic acid to conditions sufficient to convert vanillic acid to 4-amino-3-methoxybenzoic acid; (c) subjecting 4-amino-3-methoxybenzoic acid to conditions sufficient to convert 4-amino-3-methoxybenzoic acid to 2-aminophenol; a subjecting 2-aminophenol to conditions sufficient to produce aniline; and aniline obtained thereby.

Also provided herein is a method of obtaining o-phenylenediamine, comprising: (a) subjecting lignin to oxidative conditions sufficient to produce vanillic acid, (b) subjecting vanillic acid to conditions sufficient to convert vanillic acid to 4-amino-3-methoxybenzoic acid; and (c) subjecting 4-amino-3-methoxybenzoic acid to conditions sufficient to convert 4-amino-3-methoxybenzoic acid to OPDA; and o-phenylenediamine obtained thereby.

Provided in some embodiments herein is a method of obtaining caprolactam, comprising: ((a) subjecting lignin to oxidative conditions sufficient to produce vanillic acid, (b) subjecting vanillic acid to conditions sufficient to convert vanillic acid to cyclohexanecarboxylic acid; and (d) subjecting cyclohexanecarboxylic acid to conditions sufficient to convert cyclohexanecarboxylic acid to caprolactam; and caprolactam obtained thereby.

In some embodiments, in any of the methods described herein, the step of subjecting lignin to oxidative conditions sufficient to produce vanillic acid comprises (1) contacting lignin with a catalytic amount of nitroarene catalyst to oxidize lignin and provide an inactive nitroarene byproduct; and (2) recycling the nitroarene byproduct to regenerate the nitroarene catalyst. In certain embodiments, recycling the nitroarene byproduct to regenerate the nitroarene catalyst comprises contacting the nitroarene byproduct with molecular oxygen and a metal catalyst. In other embodiments, recycling the nitroarene byproduct to regenerate the nitroarene catalyst comprises contacting the nitroarene byproduct with molecular oxygen and an N-oxygenase. In specific embodiments, the N-oxygenase is AurF from Streptomyces thioluteus.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates nitroarene byproducts produced by oxidative deconvolution of lignin with nitroarenes.

FIG. 2 illustrates the depolymerization of lignin and one embodiment of the recycling of catalytic nitroarene from nitroarene byproducts produced by the oxidative depolymerization of lignin with nitroarenes.

FIG. 3 illustrates recycling of catalytic nitroarene from nitroarene byproducts produced by the reduction of nitroarenes.

FIG. 4 illustrates the depolymerization of lignin and one embodiment of the recycling of catalytic nitroarene from nitroarene byproducts produced by the oxidative depolymerization of lignin with nitroarenes.

FIG. 5 illustrates oxidative and thermal deconvolution process for the preparation of vanillic acid.

FIG. 6 illustrates various lignin deconvolution processes and downstream processing.

FIG. 7 illustrates the aqueous and catalytic deconvolution process for the preparation of catechol.

### DETAILED DESCRIPTION OF THE INVENTION

### I. Certain Definitions

Unless defined otherwise, all technical and scientific terms used herein have the same meaning as is commonly understood by one of skill in the art to which the claimed subject matter belongs.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, (e.g., “include”, “includes”, and “included”) is not limiting.

As used herein, “purified” refers to lignin which is at least 50% pure, at least 60% pure, at least 70% pure, at least 75% pure, at least 80% pure, at least 85% pure, at least 90% pure, at least 95% pure, at least 99% or greater pure.

As used herein, “biomass” means biological material that is used for industrial production (e.g., to generate chemical compounds). In some embodiments, biomass is virgin biomass, non-virgin biomass (e.g., agricultural biomass, commercial organics, and yard waste); or blended biomass. Biomass includes, but is not limited to, trees, shrubs, grasses, wheat, wheat straw, sugar cane bagasse, corn, corn husks, corn stover, corn kernel including fiber from kernels, products and by-products from milling of grains such as corn (including wet milling and dry milling). In some embodiments, biomass is used as collected from the field. In some embodiments, biomass is processed, for example by milling, grinding, shredding, etc. In some embodiments, biomass is treated by chemical or physical means prior to use, for example by treating with acid, treating with base, heating, drying, freezing, or by ensiling (storing for period of time at high moisture content).

As used herein, “agricultural biomass” includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, vines, and hard and soft woods (not including woods with deleterious materials). Generally, the substrate is of high lignocellulose content, including corn stover, corn fiber, Distiller's dried grains, rice straw, hay, sugarcane bagasse, wheat, oats, barley malt and other agricultural biomass, switchgrass, forestry wastes, poplar wood chips, pine wood chips, sawdust, yard waste, and the like, including any combination of substrate.

As used herein, “blended biomass” means any mixture or blend of virgin and non-virgin biomass, preferably having about 5-95% by weight non-virgin biomass.

As used herein, “lignin” means a water-insoluble macromolecule comprised of three monolignol monomers: p-coumaryl alcohol, coniferyl alcohol, and sinapyl alcohol.

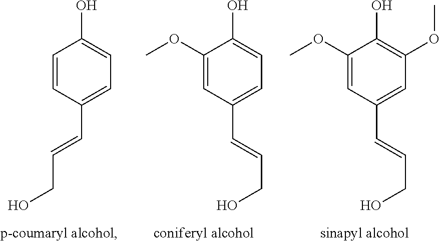

In certain instances, p-coumaryl alcohol, coniferyl alcohol and sinapyl alcohol randomly assemble to form the copolycondensate lignin. In certain instances, lignin further comprises additional monomeric units. In certain instances, lignin comprises about 15% to about 30% the weight of dry wood. In some embodiments, lignin is isolated from a lignin source (i.e., any source of lignin; e.g., unprocessed biomass and processed biomass). In some embodiments, lignin is not isolated from a lignin source.

As used herein, “cellulose” means a polysaccharide consisting of a linear chain of about 7,000 to about 15,000 β(1→4) linked D-glucose units. In certain instances, cellulose comprises about 30% to about 50% the weight of dry wood.

As used herein, “hemicellulose” means a heteropolymer consisting of a branched chain of about 500 to about 3000 sugar (e.g., glucose, xylose, mannose, galactose, rhamnose, and arabinose) units. In certain instances, hemicellulose comprises about 10% to about 35% the weight of dry wood.

The terms “ligninase” or “lignin-modifying enzymes” are interchangeable. As used herein, the terms mean an enzyme that catalyzes the breakdown of lignin. In various instances, the enzymes may be oxidative enzymes. In certain instances, the enzymes may be extracellular fungal enzymes, fungal enzymes, bacterial enzymes, or the like. Ligninase includes, but is not limited to, peroxidases (e.g., Lignin peroxidase, Manganese peroxidase and Versatile peroxidase), and phenol-oxidases of Laccase type. Fungi that produce ligninase include, but are not limited to, Phanerochaete chrysosporium, Ceriporiopsis subvermispora, Trametes versicolor, Phlebia radiata, Pleurotus ostreatus, Pleurotus eryngii, and Agaricus bisporus. Bacteria that produce ligninase include, but are not limited to, Streptomyces viridosporus T7A, Streptomyces lavendulae REN-7 and Clostridium stercorarium. In some embodiments, the ligninase has been post-translationally modified. In some embodiments, a ligninase is produced by fermentation methods and/or recombinant methods. In some embodiments, the ligninase is immobilized.

The terms “carboxy-lyase” and “decarboxylase” are used interchangeably. As used herein, the terms mean a carbon-carbon lyase that adds or removes a carboxyl group from organic compounds. In some embodiments, a decarboxylase is produced by fermentation methods and/or recombinant methods. In some embodiments, a decarboxylase is derived from Streptomyces sp., Bacillus megaterium (CAS 68038-67-5), or a combination thereof. In some embodiments, the decarboxylases are modified forms of known or naturally-occurring decarboxylases and/or have been selected from a library of naturally-occurring and/or modified decarboxylases. In some embodiments, the decarboxylase has been “post-translationally modified.” In some embodiments, the decarboxylase is immobilized.

As used herein, the term “catechol dioxygenase” means a metalloprotein enzyme that catalyzes the oxidative cleavage of catechols. Catechol dioxygenases have several different substrate specificities, including catechol 1,2-dioxygenase (EC 1.13.11.1), catechol 2,3-dioxygenase (EC 1.13.11.2), and protocatechuate 3,4-dioxygenase (EC 1.13.11.3). In some embodiments, the catechol dioxygenase is an isolated catechol dioxygenase. In some embodiments, catechol dioxygenase is selected from a catechol dioxygenase of Pseudomonas putida, Escherichia coli, Pseudomonas arvill (e.g., ATCC 23974), Pseudomonas aeruginosa, Acinetobacter sp., Arthrobacter sp., and combinations thereof. In some embodiments, the catechol dioxygenase has been isolated from cell culture in which it has been produced by fermentation. In further or alternative embodiments, the catechol dioxygenase has been produced in a recombinant host cell. In further or alternative embodiments, the catechol dioxygenase is a modified form of a naturally occurring enzyme. In some embodiments, the catechol dioxygenase has been “post-translationally modified.” In some embodiments, the catechol dioxygenase is immobilized.

As used herein, “post-translationally modified” means any modification of an amino acid which occurs after such an amino acid has been translationally incorporated into a polypeptide chain. Such modifications include, but are not limited to, co-translational in vivo modifications, co-translational in vitro modifications (such as in a cell-free translation system), post-translational in vivo modifications, and post-translational in vitro modifications.

As used herein, “pure” means a substance that is substantially free of other components.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic. Furthermore, the alkyl moiety, whether saturated or unsaturated, may comprise branched, straight chain, and/or cyclic portions. Depending on the structure, an alkyl group can be a monoradical or a diradical (i.e., an alkylene group). A “heteroalkyl” group is as described for “alkyl” with at least one of the C atoms thereof substituted with an N, S, or O atom. The “heteroalkyl” group may comprise linear, branched, and/or cyclic portions. In certain embodiments, a “lower alkyl” is an alkyl group with 1-6 carbon atoms (i.e., a C1-C6 alkyl group). In specific instances, the “lower alkyl” may be straight chained or branched.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Illustrative examples of cycloalkyl groups include the following moieties:

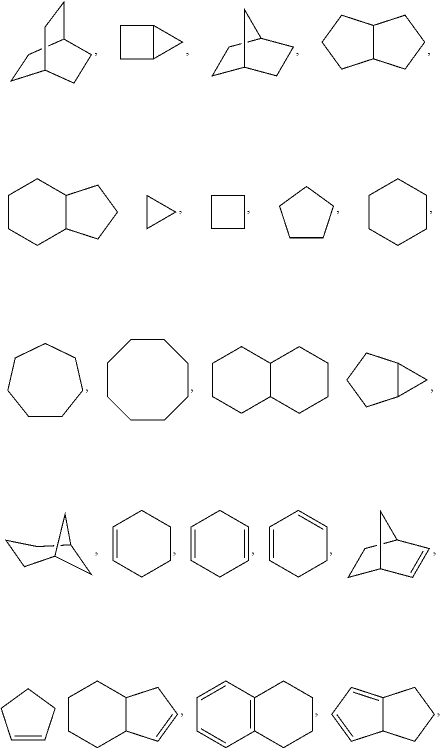

and the like.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

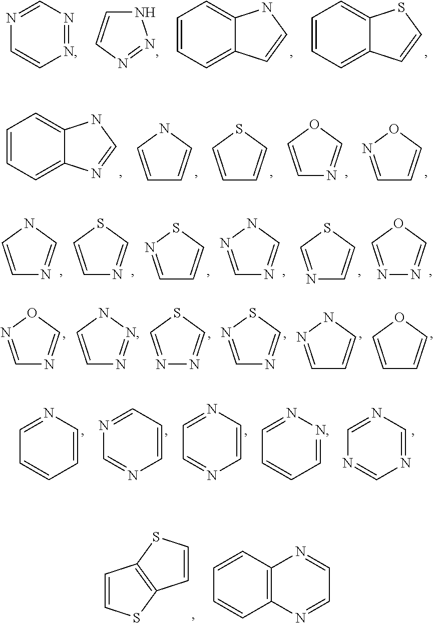

and the like. Depending on the structure, a heteroaryl group can be a monoradical or a diradical (i.e., a heteroarylene group).

A “heterocycloalkyl” group refers to a cycloalkyl group that includes at least one ring atom that is not a carbon, i.e. at least one ring atom is a heteroatom selected from nitrogen, oxygen and sulfur. The heterocycloalkyl radicals may be fused with an aryl or heteroaryl. Illustrative examples of heterocycloalkyl groups, also referred to as non-aromatic heterocycles, include:

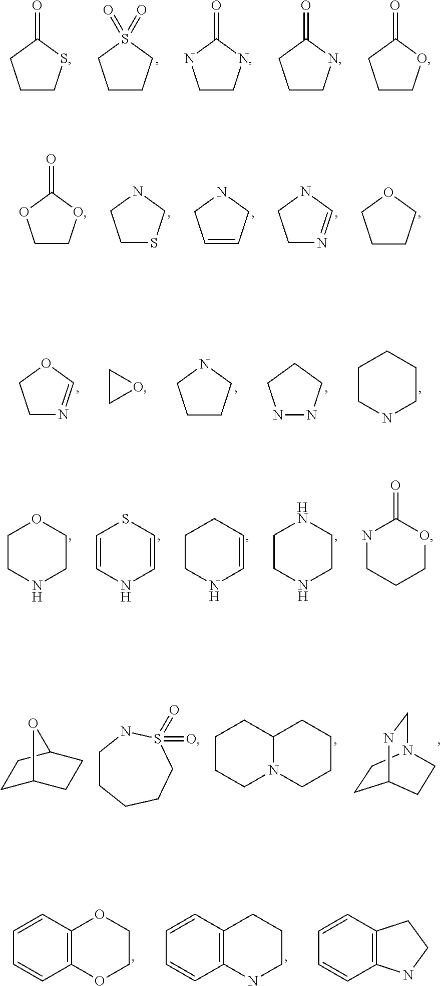

and the like. The term heteroalicyclic also includes all ring forms of the carbohydrates, including but not limited to the monosaccharides, the disaccharides and the oligosaccharides. Heterocycloalkyls have from 2 to 10 carbons in the ring. A “lower heterocycloalkyl” has 2 to 8 ring carbon atoms. It is understood that when referring to the number of carbon atoms in a heterocycloalkyl, the number of carbon atoms in the heterocycloalkyl is not the same at the total number of atoms (including the heteroatoms) that make up the heterocycloalkyl (i.e skeletal atoms of the heterocycloalkyl ring).

The term “modulate,” as used herein refers to having some affect on (e.g., increasing, enhancing or maintaining a certain level).

The term “optionally substituted” or “substituted” means that the referenced group may be substituted with one or more additional group(s) individually and independently selected from C₁-C₆alkyl, C₃-C₈cycloalkyl, aryl, heteroaryl, C₂-C₆heteroalicyclic, hydroxy, C₁-C₆alkoxy, aryloxy, C₁-C₆alkylthio, arylthio, C₁-C₆alkylsulfoxide, arylsulfoxide, C₁-C₆alkylsulfone, arylsulfone, cyano, halo, C₂-C₈acyl, C₂-C₈acyloxy, nitro, C₁-C₆haloalkyl, C₁-C₆-fluoroalkyl, and amino, including C₁-C₆alkylamino, and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)O—, —(C₁-C₆alkyl)-, or —(C₂-C₆alkenyl)-; and each R^{s} is independently selected from H, (C₁-C₄alkyl), (C₃-C₈cycloalkyl), heteroaryl, aryl, and C₁-C₆heteroalkyl. Optionally substituted non-aromatic groups may be substituted with one or more oxo (═O). The protecting groups that may form the protective derivatives of the above substituents are known to those of skill in the art and may be found in references such as Greene and Wuts, above. In some embodiments, alkyl groups described herein are optionally substituted with an 0 that is connected to two adjacent carbon atoms (i.e., forming an epoxide).

### II. Lignin Source

In some embodiments, the lignin is obtained from any suitable source. In some embodiments, the lignin is obtained from biomass. In some embodiments, lignin is obtained from biomass comprising a lignin-carbohydrate complex. In some embodiments, the lignin is obtained from lignocellulose. In some embodiments, the lignin is obtained from biomass that has been treated by a pretreatment process of the paper, pulp or biofuel industries (e.g., Kraft process, acid hydrolysis, steam explosion, ammonia fiber explosion, ammonia recycle percolation, soaking in aqueous ammonia, lime (with or without oxygen) treatment, alkaline wet oxidation and ozonolysis). In some embodiments, the lignin is purified lignin. In certain embodiments, the lignin is substantially intact lignin.

In some embodiments, the lignin utilized in a method described herein is substantially intact. As used herein, “substantially intact” means that the lignin comprises coumarylalcohol, coniferylalcohol and sinapylalcohol condensation moieties that have not been substantially or significantly modified by a process described herein (e.g., the coumarylalcohol, coniferylalcohol and sinapylalcohol condensation moieties of the lignin are substantially in their natural state).

In some embodiments, the lignin utilized in a method described herein is substantially free of cellulose, hemicellulose, residues or fragments thereof.

### III. Oxidation of Lignin into Vanillic Acid

Disclosed herein, in certain embodiments, is vanillic acid obtained from lignin. Further disclosed herein, in certain embodiments, is a method of obtaining vanillic acid from lignin. It is to be understood that provided in various embodiments herein are both (1) vanillic acid prepared according to any of the processes described herein for preparing vanillic acid from a lignin source; and (2) any process described herein for preparing vanillic acid from a lignin source.

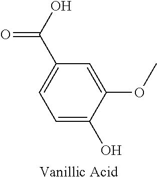

Disclosed herein, in certain embodiments, is a method of obtaining vanillic acid from lignin, comprising: subjecting lignin to oxidative conditions sufficient to produce vanillic acid. In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating a lignin containing combination includes, by way of non-limiting example, heating to at least room temperature, at least 25° C., at least 35° C., at least 50° C., at least 70° C., at least 80° C., at least 90° C., or at least 100° C.

### A. Enzymatic Oxidation

Disclosed herein, in certain embodiments, is a method of obtaining vanillic acid from lignin, comprising: subjecting lignin to oxidative conditions sufficient to produce vanillic acid. In some embodiments, the method comprises contacting lignin with a ligninase. In some embodiments, the enzymatic reaction occurs under conditions that instigate or maximize the reaction (e.g., heating or high pressure). In some embodiments, the ligninase is an oxidative ligninase. In some embodiments, the ligninase is a peroxidase, an oxidase, or combinations thereof. In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating a lignin containing combination includes, by way of non-limiting example, heating to at least room temperature, at least 25° C., at least 35° C., at least 50° C., at least 70° C., at least 80° C., at least 90° C., or at least 100° C.

In some embodiments, the peroxidase is an isolated peroxidase. In some embodiments, the peroxidase is produced by fermentation methods and/or recombinant methods. In some embodiments, the peroxidase is a modified form of a known or naturally-occurring peroxidase and/or has been selected from a library of naturally-occurring and/or modified peroxidases.

In some embodiments, the oxidase is an isolated oxidase. In some embodiments, the oxidase is produced by fermentation methods and/or recombinant methods. In some embodiments, the oxidase is a modified form of a known or naturally-occurring oxidase and/or has been selected from a library of naturally-occurring and/or modified oxidases.

### B. Use of an Oxidizing Agent

Disclosed herein, in certain embodiments, is a method of obtaining vanillic acid from lignin, comprising: subjecting lignin to oxidative conditions sufficient to produce vanillic acid. In some embodiments, the method comprises contacting lignin with an oxidizing agent. In some embodiments, the oxidizing agent is hydrogen peroxide (H₂O₂). In some embodiments, the oxidizing agent is molecular oxygen (i.e., O₂). In certain embodiments, the oxidizing agent is a nitroarene, such as nitrobenzene (e.g., catalytic nitrobenzene) or substituted nitrobenzene. In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating a lignin containing combination includes, by way of non-limiting example, heating to at least room temperature, at least 25° C., at least 35° C., at least 50° C., at least 70° C., at least 80° C., at least 90° C., or at least 100° C.

In specific embodiments, the oxidizing agent is a nitroarene. The use of non-catalytic amounts of nitroarene in oxidizing lignin can be commercially cost prohibitive. Thus, in some embodiments, the oxidizing agent is catalytic nitroarene (e.g., catalytic nitrobenzene and/or substituted nitrobenzene). Oxidation of lignin produces inactive (i.e., reduced) nitroarene byproducts. FIG. 1 illustrates the reduction of nitrobenzene and various byproducts resulting from use of such oxidants. In some embodiments, e.g., wherein catalytic amounts of nitroarene (e.g., nitrobenzene and/or substituted nitrobenzene) are utilized, a method described herein further comprises recycling these inactive nitroarene byproducts, e.g., by re-oxidizing the reduced nitroarene byproducts to an active oxidizing agent, e.g., a catalytic nitroarene. As a result, this recycling process now provides for the use of nitroarenes as catalysts.

In certain embodiments, the nitroarene oxidizes lignin and is recycled in a single pot. In some embodiments, the nitroarene byproducts are removed from the lignin oxidation system, re-oxidized, and returned to the lignin oxidation system. In certain embodiments, nitroarene oxidation of lignin is performed at ambient or low pressure (e.g., less than 5 atm, less than 3 atm, less than 2 atm, or less than 1.2 atm). In some embodiments, nitroarene oxidation of lignin is performed at ambient or low temperature (e.g., less than 80° C., less than 50° C., or less than 30° C.). In some embodiments, an additional additive is added to the lignin/nitroarene combination. In certain embodiments, the additive is an additive for improving aldehyde yields (e.g., an anthraquinone).

In certain embodiments, the nitroarene byproducts are re-oxidized utilizing any suitable system.

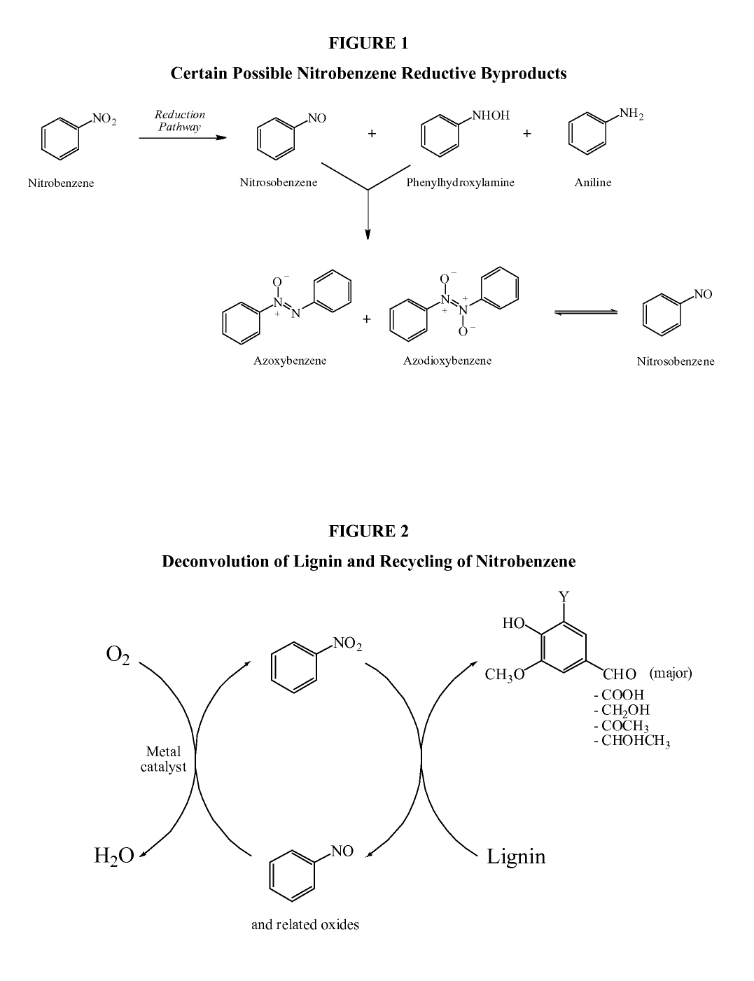

In some embodiments, the nitroarene byproducts are re-oxidized utilizing an oxidizing agent (e.g., molecular oxygen) and a metal catalyst (e.g., a molybdenum catalyst, a rhenium catalyst, a tungsten catalyst, a cobalt (e.g., Co²⁺) catalyst, or the like). FIG. 2 illustrates oxidation of lignin and recycling of nitroarene catalysts according to one embodiment of a process described herein. Specifically, FIG. 2 illustrates an electron transport from lignin to, eventually, molecular oxygen. In specific embodiments, the metal catalyst is utilized in an amount of less than 5 mol % (e.g., compared to the amount of nitroarene catalyst utilized). In more specific embodiments, the metal catalyst is utilized in an amount of less than 1 mol % (e.g., compared to the amount of nitroarene catalyst utilized). In still more specific embodiments, the metal catalyst is utilized in an amount of less than 0.1 mol % (e.g., compared to the amount of nitroarene catalyst utilized). In other specific embodiments, the metal catalyst is utilized in an amount of 0.01 mol % to about 1 mol % (e.g., compared to the amount of nitroarene catalyst utilized). In certain embodiments, re-oxidation or recycling of nitroarene catalytic byproducts is performed at ambient or low pressure (e.g., less than 5 atm, less than 3 atm, less than 2 atm, or less than 1.2 atm). In some embodiments, re-oxidation or recycling of nitroarene catalytic byproducts is performed at ambient or low temperature (e.g., less than 80° C., less than 50° C., or less than 30° C.). In certain embodiments, provided herein is a composition comprising lignin, nitroarene, and a metal catalyst. In some embodiments, the composition further comprises any compound of Formula I:

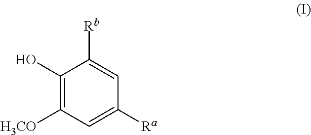

wherein R^{a} is CHO, COOH, CH₂OH, COCH₃, or CHOHCH₃; and R^{b} is H, OCH₃, or COOH.

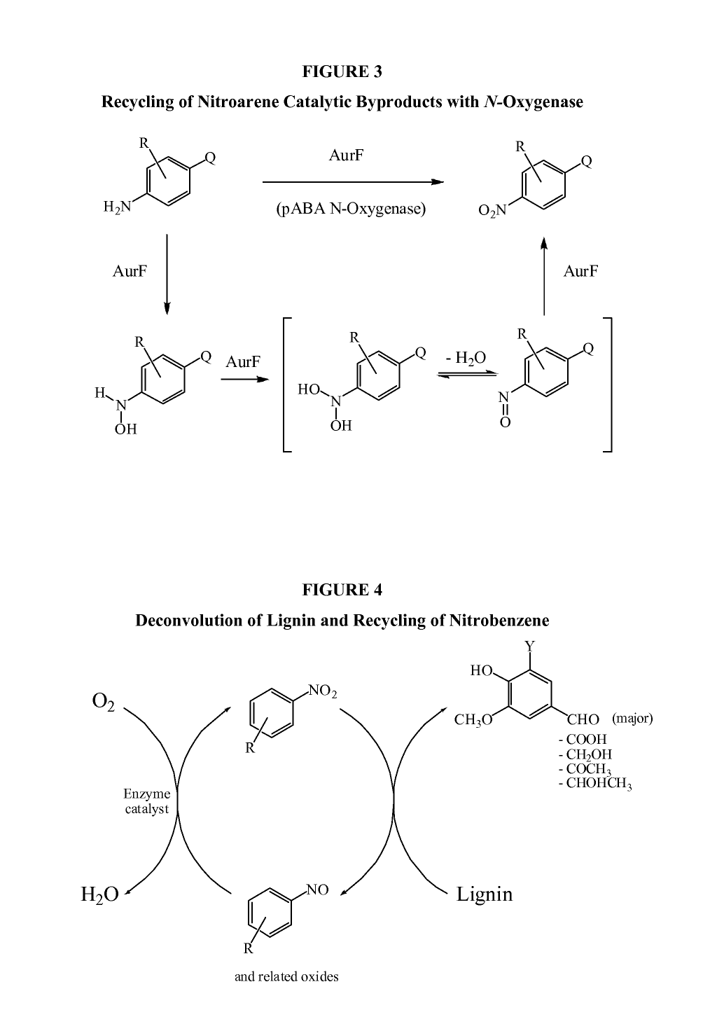

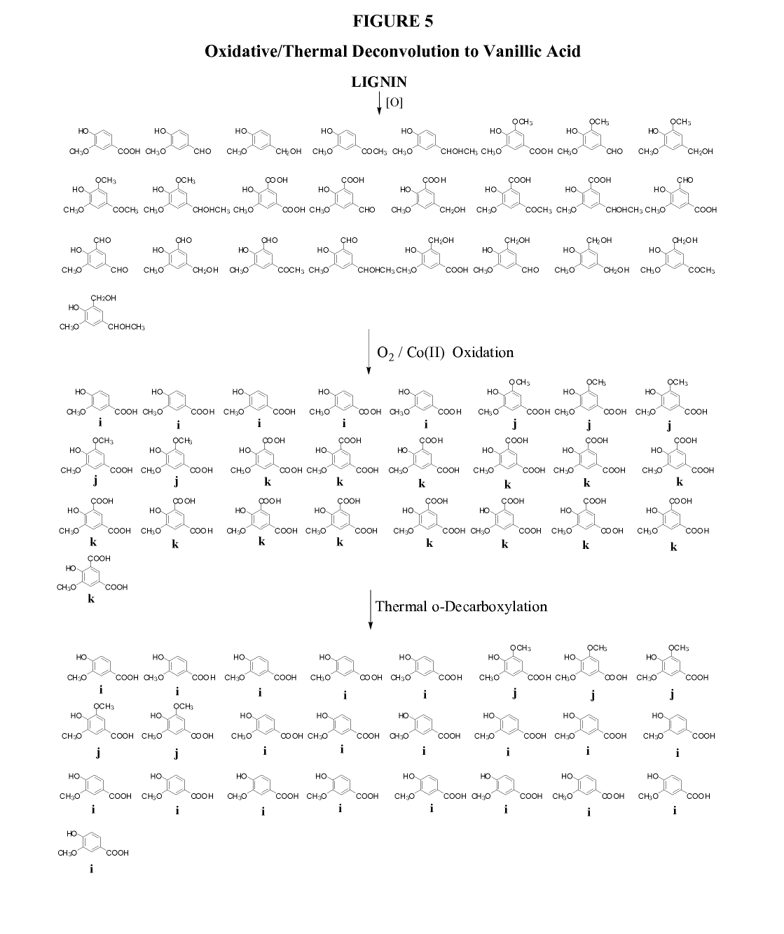

In some embodiments, the nitroarene catalytic byproducts are re-oxidized by contacting the nitroarene byproducts with an N-oxygenase (e.g., the N-oxygenase AurF, such as from Streptomyces thioluteus). In some embodiments, an oxidizing agent, such as molecular oxygen (e.g., air), is utilized in combination with the N-oxygenase, e.g., as a terminal oxidant of the catalytic nitroarene recycling process. FIG. 3 illustrates recycling of nitroarene catalytic byproducts with AurF. FIG. 4 illustrates oxidation of lignin and recycling of nitroarene catalysts according to one embodiment of a process described herein. Specifically, FIG. 4 illustrates an electron transport from lignin to, eventually, molecular oxygen. In certain embodiments, re-oxidation or recycling of nitroarene catalytic byproducts is performed at ambient or low pressure (e.g., less than 5 atm, less than 3 atm, less than 2 atm, or less than 1.2 atm). In some embodiments, re-oxidation or recycling of nitroarene catalytic byproducts is performed at ambient or low temperature (e.g., less than 80° C., less than 50° C., or less than 30° C.). In certain embodiments, provided herein is a composition comprising lignin, nitroarene, and an N-oxygenase (e.g., AurF). In some embodiments, the composition further comprises any or more compound of Formula I.

In certain embodiments, a nitroarene group described herein is a compound of Formula II:

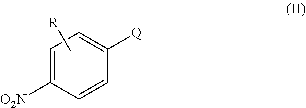

In some embodiments, R is 0-4 substituents, each of which independently are, by way of non-limiting example, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, cyano, nitro, hydroxy, thiol, arylthio, C₁-C₆alkylsulfoxide, arylsulfoxide, C₁-C₆alkylsulfone, arylsulfone, cyano, halo, C₂-C₈acyl, C₂-C₈acyloxy, C₁-C₆haloalkyl, C₁-C₆-fluoroalkyl, and amino, including C₁-C₆alkylamino, or L¹R¹, wherein each L¹ is independently selected from a bond, —O—, —C(═O)O, —C(═O)—, —O(C═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)O—, —(C₁-C₆alkyl)-, or —(C₂-C₆alkenyl)-; and each R¹ is independently selected from H, (C₁-C₄alkyl), (C₃-C₈cycloalkyl), heteroaryl, aryl, and C₁-C₆heteroalkyl. In some embodiments, Q is, by way of non-limiting example, H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, cyano, nitro, hydroxy, thiol, arylthio, C₁-C₆alkylsulfoxide, arylsulfoxide, C₁-C₆alkylsulfone, arylsulfone, cyano, halo, C₂-C₈acyl, C₂-C₈acyloxy, C₁-C₆haloalkyl, C₁-C₆-fluoroalkyl, and amino, including C₁-C₆alkylamino, or L^{q}R^{q}, wherein each L^{q} is independently selected from a bond, —O—, —C(═O)O, —C(═O)—, —O(C═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)O—, —(C₁-C₆alkyl)-, or —(C₂-C₆alkenyl)-; and each R^{q} is independently selected from H, (C₁-C₄alkyl), (C₃-C₈cycloalkyl), heteroaryl, aryl, and C₁-C₆heteroalkyl. In some embodiments, Q is a hydrogen bond donor group. In certain embodiments, R is an electron withdrawing or releasing group.

In some embodiments, the method further comprises contacting lignin with a metal catalyst. In some embodiments, the metal catalyst is a transition metal. In some embodiments, the metal catalyst is a Group VII transition metal. In some embodiments, the metal catalyst is Co, Rh, Ir, or combinations thereof. In a specific embodiment, the catalyst is a Cobalt catalyst (e.g., a Co(II) catalyst). In some embodiments, the metal catalyst is a Group VA metal. In some embodiments, the metal catalyst is Bi. In some embodiments, the metal catalyst is methyltrioxorhenium (VII) (MeReO₃).

Disclosed herein, in certain embodiments, is a method of obtaining vanillic acid from lignin, comprising: subjecting lignin to oxidative conditions sufficient to produce vanillic acid. In some embodiments, the method comprises contacting lignin with hydrogen peroxide and MeReO₃. In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating a lignin containing combination includes, by way of non-limiting example, heating to at least room temperature, at least 25° C., at least 35° C., at least 50° C., at least 70° C., at least 80° C., at least 90° C., or at least 100° C.

### C. Improved Yield

In certain embodiments, a process described herein comprises preparing vanillic acid from lignin, whereby vanillic acid is prepared from the lignin in a yield of about 5% to about 60% by weight of the lignin. In some embodiments, vanillic acid is prepared from the lignin in a yield of about 15% to about 50% by weight of the lignin. FIG. 5 illustrates a process of producing vanillic acid from lignin in high yield.

In some embodiments, lignin is oxidized to a combination of aromatic compounds (e.g., as illustrated in FIG. 5) by use of an oxidizing system comprising, e.g., an enzyme (e.g., ligninase, peroxidase, oxidase, or combination thereof); a metal catalyst; nitroarene (e.g., nitrobenzene); oxygen; peroxide; or a combination thereof. In specific embodiments, lignin is oxidized with nitroarene (e.g., nitrobenzene and/or substituted nitrobenzene). The combination of aromatic compound obtained by oxidizing lignin can then be further oxidized, and optionally thermally o-decarboxylated, to convert a number of different aromatic compounds in the combination of aromatic compounds to one or more like compounds. FIG. 5 illustrates a specific embodiment of this process, i.e., oxidation of the combination of aromatic compounds obtained by oxidizing lignin with molecular oxygen and catalytic Co(II), and optionally thermally o-decarboxylating, e.g., by thermal mechanism. In other embodiments, other oxidizing agent(s) and optional catalysts are utilized. For example, in certain embodiments, the combination of oxidative compounds are further oxidized using an enzyme (e.g., ligninase, peroxidase, oxidase, or combination thereof); a metal catalyst; oxygen; peroxide; or a combination thereof, as illustrated in Scheme 1:

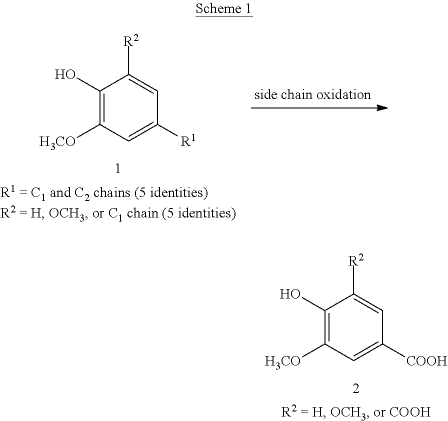

In specific embodiments, provided herein is a compound or mixture of compounds of Formula 2, e.g., as produced by Scheme 1. In certain embodiments, vanillic acid prepared according to scheme 1 is at least 50%, at least 60%, at least 70%, at least 80%, at least 85%, at least 90%, at least 95%, or at least 98% free of compound of Formula 2 having R²═OCH₃ or COOH. In some embodiments, provided herein is a vanillic acid composition comprising (i) 50-98% w/w vanillic acid, and (ii) 1-30% w/w compound of Formula 2, wherein R² is OCH₃ and/or 1-30% w/w compound of Formula 2, wherein R² is COOH. In specific embodiments, the vanillic acid composition comprises 2-15% w/w compound of Formula 2, wherein R² is OCH₃ and/or 2-15% w/w compound of Formula 2, wherein R² is COOH. In some embodiments, the yield of vanillic acid is further improved by o-decarboxylating (e.g., thermally) the compound of Formula 2 wherein R² is COOH.

### D. Isolation of Vanillic Acid

In some embodiments, the vanillic acid is isolated from the remaining lignin. In some embodiments, vanillic acid is isolated by any method suitable.

In some embodiments, the vanillic acid is separated from the lignin by use of a solvent. In some embodiments, the vanillic acid is separated from the lignin by use of a solvent in which the vanillic acid is soluble and the lignin is either insoluble or sparingly soluble. In some embodiments, the vanillic acid is separated from the lignin by use of a solvent in which vanillic acid is either insoluble or sparingly soluble and lignin is soluble. In some embodiments, the solvent and the soluble components are separated from the insoluble components by any suitable method (e.g., filtration). Where the vanillic acid is soluble, in some embodiments, the vanillic acid is precipitated from the solvent. In some embodiments, the method comprises washing the vanillic acid with a second solvent to further remove impurities. In some embodiments, the solvent with which the purified vanillic acid is extracted is a tunable solvent, such as a gas-expanded liquid.

In some embodiments, the vanillic acid/residual lignin material is washed with a first solvent in which vanillic acid is either insoluble or sparingly soluble and subsequently washed with a second solvent in which vanillic acid is soluble.

In some embodiments, (a) vanillic acid and the residual lignin material are washed with a first solvent; (b) the first solvent is removed (e.g., by evaporation or filtration); and (c) the vanillic acid is further purified (i) by washing the vanillic acid with a second solvent in which the vanillic acid is insoluble (or sparingly soluble) and in which at least some of the contaminants are at least partially soluble, (ii) by dissolving the vanillic acid in a second solvent in which at least some of the contaminants are insoluble (or sparingly soluble), and/or (iii) by dissolving the vanillic acid and at least some of the contaminants in a second solvent and selectively precipitating vanillic acid and/or the various contaminants from the second solvent.

### E. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### IV. Further Products

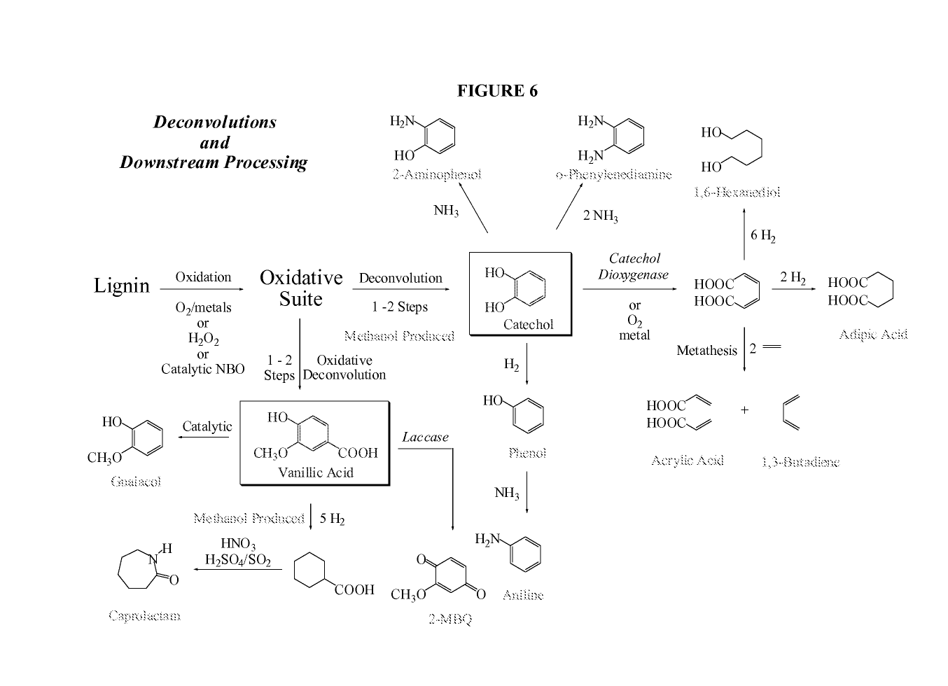

In various embodiments, provided herein is a process of preparing any compound described in FIG. 6, or a compound prepared according to such a process.

### A. 2-Methoxyphenol

Disclosed herein, in certain embodiments, is 2-methoxyphenol obtained from lignin. Further disclosed herein, in certain embodiments, is a method of obtaining 2-methoxyphenol from lignin. It is to be understood that provided in various embodiments herein are both (1) 2-methoxyphenol prepared according to any of the processes described herein; and (2) any method described herein for obtaining 2-methoxyphenol from a lignin source.

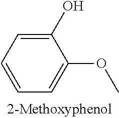

Disclosed herein, in certain embodiments, is a method of obtaining 2-methoxyphenol from lignin, comprising: (a) subjecting lignin to oxidative conditions sufficient to produce vanillic acid, and (b) subjecting vanillic acid to conditions sufficient to convert vanillic acid to 2-Methoxyphenol. In some embodiments, lignin is converted to vanillic acid by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of Vanillic Acid to 2-Methoxyphenol

In some embodiments, converting vanillic acid to 2-methoxyphenol comprises decarboxylating vanillic acid. In some embodiments, decarboxylating vanillic acid comprises contacting vanillic acid with a decarboxylase under conditions that instigate or maximize the enzymatic reaction. In some decarboxylating vanillic acid comprises contacting vanillic acid with a decarboxylase and heating the combination. In some embodiments, the decarboxylase/vanillic acid combination is heated to about 200° C., about 250° C., about 300° C., about 350° C., or about 350° C. In some embodiments, decarboxylating vanillic acid comprises contacting vanillic acid with super critical or near critical water.

ii. Isolation of 2-Methoxyphenol

In some embodiments, the 2-methoxyphenol is isolated from the remaining vanillic acid. In some embodiments, 2-methoxyphenol is isolated by any method suitable.

In some embodiments, the 2-methoxyphenol is isolated from the remaining vanillic acid by use of a solvent. In some embodiments, the 2-methoxyphenol is isolated from the remaining vanillic acid by use of a solvent in which the vanillic acid is soluble and the 2-methoxyphenol is either insoluble or sparingly soluble. In some embodiments, the vanillic acid is separated from the 2-methoxyphenol by use of a solvent in which vanillic acid is either insoluble or sparingly soluble and 2-methoxyphenol is soluble. In some embodiments, the solvent and the soluble components are separated from the insoluble components by any suitable method (e.g., filtration). Where the 2-methoxyphenol is soluble, in some embodiments, the 2-methoxyphenol is precipitated from the solvent. In some embodiments, the method comprises washing the 2-methoxyphenol with a second solvent to further remove impurities. In some embodiments, the solvent with which the purified 2-methoxyphenol is extracted is a tunable solvent, such as a gas-expanded liquid.

In some embodiments, the vanillic acid/2-methoxyphenol combination is washed with a first solvent in which 2-methoxyphenol is either insoluble or sparingly soluble and subsequently washed with a second solvent in which 2-methoxyphenol is soluble.

In some embodiments, (a) 2-methoxyphenol and the residual vanillic acid material are washed with a first solvent; (b) the first solvent is removed (e.g., by evaporation or filtration); and (c) the 2-methoxyphenol is further purified (i) by washing the 2-methoxyphenol with a second solvent in which the 2-methoxyphenol is insoluble (or sparingly soluble) and in which at least some of the contaminants are at least partially soluble, (ii) by dissolving the 2-methoxyphenol in a second solvent in which at least some of the contaminants are insoluble (or sparingly soluble), and/or (iii) by dissolving the 2-methoxyphenol and at least some of the contaminants in a second solvent and selectively precipitating 2-methoxyphenol and/or the various contaminants from the second solvent.

iii. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### B. Catechol

Disclosed herein, in certain embodiments, catechol is obtained from lignin. Further disclosed herein, in certain embodiments, is a method of obtaining catechol from lignin. It is to be understood that provided in various embodiments herein are both (1) catechol prepared according to any of the processes described herein; and (2) any method described herein for obtaining catechol from a lignin source.

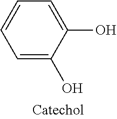

Disclosed herein, in certain embodiments, is a method of obtaining catechol from lignin, comprising: (a) subjecting lignin to oxidative conditions sufficient to produce vanillic acid, and (b) subjecting vanillic acid to conditions sufficient to convert vanillic acid to catechol. In some embodiments, lignin is converted to vanillic acid by any method disclosed herein. In some embodiments, vanillic acid is converted to 2-methoxyphenol by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of Vanillic Acid to Catechol

In some embodiments, converting vanillic acid to catechol comprises (a) decarboxylating vanillic acid such that 2-methoxyphenol is produced; and (b) contacting the 2-methoxyphenol with supercritical or near critical water to demethylate 2-methoxyphenol. As used herein, “supercritical water” is water heated to about 400° C. and at about 23 MPa. As used herein, “near critical water” is water that has a temperature and/or pressure about 1% less than, about 2% less than, about 3% less than, about 5% less than, about 10% less than, or about 15% less than is necessary for the water to become supercritical.

In some embodiments, decarboxylating vanillic acid comprises contacting vanillic acid with a decarboxylase under conditions that instigate or maximize the enzymatic reaction. In some embodiments, decarboxylating vanillic acid comprises contacting vanillic acid with a decarboxylase and heating the combination. In some embodiments, the decarboxylase/vanillic acid combination is heated to about 200° C., about 250° C., about 300° C., about 350° C., or about 350° C.

In some embodiments, 2-methoxyphenol is converted to catechol without isolating 2-methoxyphenol from vanillic acid. In some embodiments, 2-methoxyphenol is isolated from vanillic acid before it is converted into catechol. In some embodiments, 2-methoxyphenol is isolated from vanillic acid by any method disclosed herein.

In some embodiments, subjecting vanillic acid to conditions sufficient to convert vanillic acid directly to catechol includes, e.g., subjecting vanillic acid to supercritical water or subjecting vanillic acid to near-critical water, both of which are as defined herein for converting 2-methoxyphenol to catechol.

In some embodiments, methanol is generated from the demethylation of 2-methoxyphenol. In some embodiments, methanol is isolated from the reaction mixture.

ii. Improved Yield

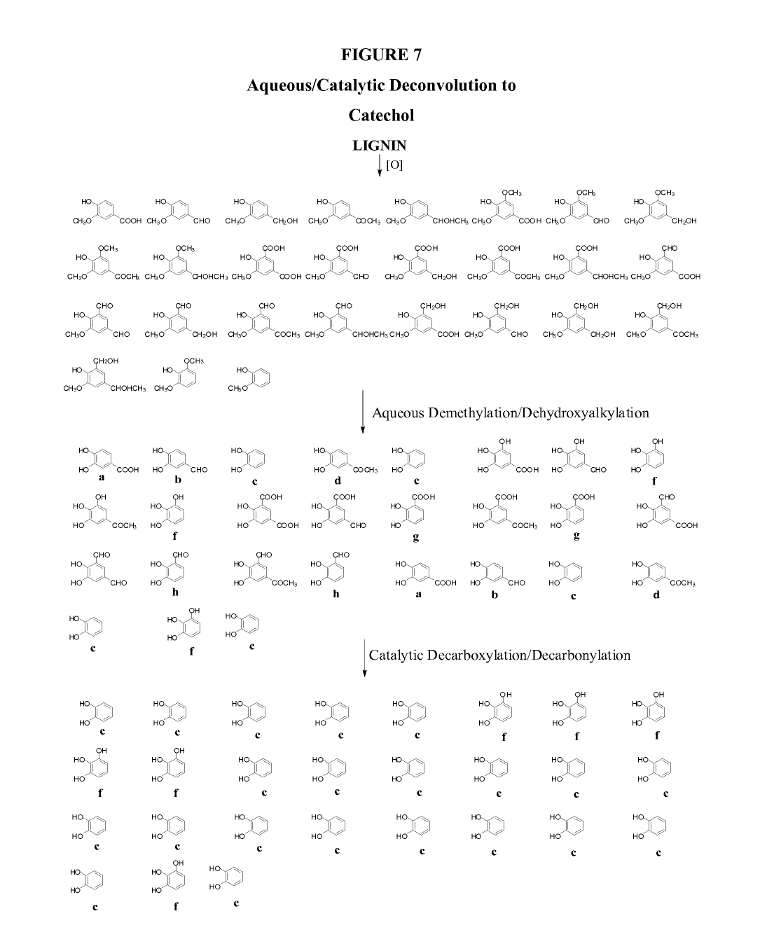

In certain embodiments, a process described herein comprises preparing catechol from lignin, whereby catechol is prepared from the lignin in a yield of about 5% to about 60% by weight of the lignin. In some embodiments, catechol is prepared from the lignin in a yield of about 15% to about 50% by weight of the lignin. FIG. 7 illustrates a process of producing catechol from lignin in high yield.

In some embodiments, lignin is oxidized to a combination of aromatic compounds (e.g., comprising any one or more of the compounds as illustrated in FIG. 7) by use of an oxidizing system comprising, e.g., an enzyme (e.g., ligninase, peroxidase, oxidase, or combination thereof); a metal catalyst; nitrobenzene; oxygen; peroxide; or a combination thereof. The combination of aromatic compound obtained by oxidizing lignin can then be subjected to aqueous demethylation and/or dehydroxylation, and optionally catalytic decarboxylation and/or decarbonylation, to convert a number of different aromatic compounds in the combination of aromatic compounds to one or more like compounds. (In some embodiments, the combination of aromatic compounds obtained from oxidizing lignin are purified, or partially purified, before further processing according to a process as illustrated in FIG. 7.) For example, in certain embodiments, the combination of oxidative compounds are further oxidized using an enzyme (e.g., ligninase, peroxidase, oxidase, or combination thereof); a metal catalyst; oxygen; peroxide; or a combination thereof, as illustrated in Scheme 2:

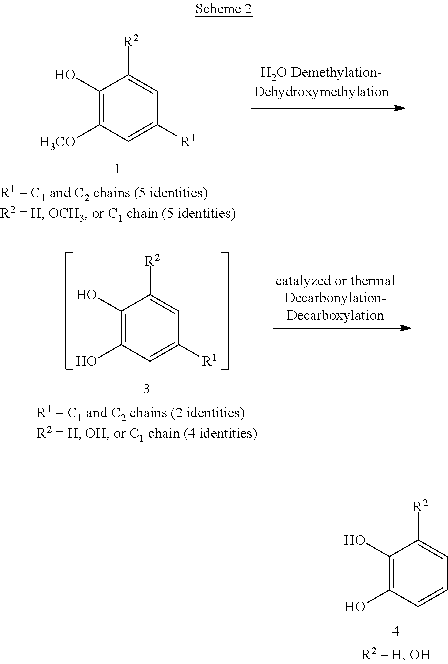

In specific embodiments, provided herein is a compound 4, e.g., as produced by Scheme 2. In certain embodiments, catechol prepared according to scheme 2 is at least 50%, at least 60%, at least 70%, at least 80%, at least 85%, at least 90%, at least 95%, or at least 98% free of compound 4 having R²═OH. In some embodiments, provided herein is a catechol composition comprising (i) 70-98% w/w catechol, and (ii) 2-30% w/w compound 4, wherein R² is OH. In specific embodiments, the catechol composition comprises 2-15% w/w compound 4.

iii. Isolation of Catechol

In some embodiments, the catechol is isolated from any remaining lignin, vanillic acid and/or 2-methoxyphenol. In some embodiments, catechol is isolated by any suitable method. In some embodiments, the catechol is isolated from the remaining lignin, vanillic acid, methanol, and/or 2-methoxyphenol by use of a solvent. In some embodiments, the catechol is isolated from any remaining lignin, vanillic acid, methanol, and/or 2-methoxyphenol by use of at least two solvents.

iv. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### C. Phenol

Disclosed herein, in certain embodiments, is catechol obtained from lignin. Further disclosed herein, in certain embodiments, is a method of obtaining catechol from lignin. It is to be understood that provided in various embodiments herein are both (1) catechol prepared according to any of the processes described herein; and (2) any method described herein for obtaining catechol from a lignin source.

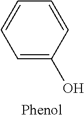

Disclosed herein, in certain embodiments, is a method of obtaining catechol from lignin, comprising: (a) subjecting lignin to oxidative conditions sufficient to produce vanillic acid, (b) subjecting vanillic acid to conditions sufficient to convert vanillic acid to catechol; and (c) subjecting catechol to conditions sufficient to convert catechol to phenol (e.g., hydroxydeoxygenation). In some embodiments, the method comprises subjecting catechol to hydroxydeoxygenation.

In some embodiments, lignin is converted to vanillic acid by any method disclosed herein. In some embodiments, vanillic acid is converted to 2-methoxyphenol by any method disclosed herein. In some embodiments, 2-methoxyphenol is converted to catechol by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of Catechol to Phenol

In some embodiments, catechol is separated or extracted from any residual material (e.g., lignin, vanillic acid, and/or 2-methoxyphenol) prior to converting it to phenol, and in other embodiments, is not separated or extracted from any residual material prior to converting it to phenol.

In some embodiments, hydroxydeoxygenation of catechol comprises contacting catechol, hydrogen, and a catalyst. In some embodiments, hydrogen and catechol are present in a ratio of molecules of hydrogen to molecules of catechol of about 2:1 to about 50:1.

In some embodiments, the catalyst is Bi or Nb. In some embodiments, the catalyst is a multicomponent metal oxide comprising a Group VB, VIB, VIII, IB, IIB, IVA and/or VA metal. In some embodiments, the catalyst has the formula Bi_{(1-x/3)}V_{(1-x)w}Nb_{(1-x)(1-w)}MoₓO₄, wherein x is between 0 and 1 and w is between 0 and 1. In some embodiments, the catalyst has the formula Cu_{(1-z)}Zn_{z}W_{(1-y)}Mo_{y}O₄, wherein y is between 0 and 1 and z is between 0 and 1. In some embodiments, the catalyst is BiVO₄, CuWO₄, Cu_{0.75}Zn_{0.25}WO₄, CuWO₄+Ce, CuO—Al₂O₃ and combinations thereof.

In some embodiments, catechol, hydrogen, and the catalyst are contacted at a temperature of about 250° C. to about 500° C. In some embodiments, catechol, hydrogen, and the catalyst are contacted at a pressure of about 1-100 bar. In some embodiments, catechol, hydrogen, and the catalyst are contacted at a temperature of about 250° C. to about 500° C. and a pressure of about 1-100 bar.

In some embodiments, methanol is generated from the demethylation of 2-methoxyphenol. In some embodiments, methanol is isolated from the reaction mixture.

ii. Isolation of Phenol

In some embodiments, the phenol is isolated from any remaining lignin, vanillic acid, methanol, 2-methoxyphenol, and/or catechol. In some embodiments, phenol is isolated by any suitable method. In some embodiments, the phenol is isolated from any remaining lignin, vanillic acid, 2-methoxyphenol, methanol, and/or catechol by use of a solvent. In some embodiments, the phenol is isolated from any remaining lignin, vanillic acid, 2-methoxyphenol, and/or catechol by use of at least two solvents.

iii. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### D. Muconic Acid

Disclosed herein, in certain embodiments, is muconic acid obtained from lignin. Further disclosed herein, in certain embodiments, is a method of obtaining muconic acid from lignin. It is to be understood that provided in various embodiments herein are both (1) muconic acid prepared according to any of the processes described herein; and (2) any method described herein for obtaining muconic acid from a lignin source.

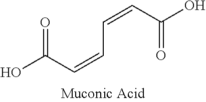

Disclosed herein, in certain embodiments, is a method of obtaining muconic acid from lignin, comprising: (a) subjecting lignin to oxidative conditions sufficient to produce vanillic acid, (b) subjecting vanillic acid to conditions sufficient to convert vanillic acid to catechol; and (c) subjecting catechol to conditions sufficient to convert catechol to muconic acid (e.g., under oxidative conditions).

In some embodiments, lignin is converted to vanillic acid by any method disclosed herein. In some embodiments, vanillic acid is converted to 2-methoxyphenol by any method disclosed herein. In some embodiments, 2-methoxyphenol is converted to catechol by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of Catechol to Muconic Acid

In some embodiments, catechol is separated or extracted from any residual material (e.g., lignin, vanillic acid, and/or 2-methoxyphenol) prior to converting it to muconic acid, and in other embodiments, is not separated or extracted from any residual material prior to converting it to muconic acid.

In some embodiments, the method comprises contacting catechol with a metal (e.g., a metal cation) or metal catalyst (e.g., a metal cation) such as, by way of non-limiting example, iron (II, III), copper (I, II) or the like. In some embodiments, the method further comprises contacting the catechol with an oxidizing agent. In some embodiments, the method further comprises contacting the catechol with O₂, H₂O₂, or combinations thereof.

In some embodiments, the method comprises contacting catechol with a catechol dioxygenase (e.g., a 1,2-catechol dioxygenase).

In some embodiments, the conversion to muconic acid occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

In some embodiments, methanol is generated from the dioxygenase induced cleavage of catechol. In some embodiments, methanol is isolated from the reaction mixture.

ii. Isolation of Muconic Acid

In some embodiments, the muconic acid is isolated from any remaining lignin, vanillic acid, 2-methoxyphenol, and/or catechol. In some embodiments, muconic acid is isolated by any suitable method. In some embodiments, muconic acid is isolated from any remaining lignin, vanillic acid, 2-methoxyphenol, methanol, and/or catechol by use of a solvent. In some embodiments, the muconic acid is isolated from any remaining lignin, vanillic acid, 2-methoxyphenol, methanol, and/or catechol by use of at least two solvents.

iii. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### E. Adipic Acid

Disclosed herein, in certain embodiments, is adipic acid obtained from lignin. Further disclosed herein, in certain embodiments, is a method of obtaining adipic acid from lignin. It is to be understood that provided in various embodiments herein are both (1) adipic acid prepared according to any of the processes described herein; and (2) any method described herein for obtaining adipic acid from a lignin source.

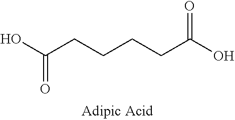

Disclosed herein, in certain embodiments, is a method of obtaining adipic acid from lignin, comprising: (a) subjecting lignin to oxidative conditions sufficient to produce vanillic acid, (b) subjecting vanillic acid to conditions sufficient to convert vanillic acid to catechol; (c) subjecting catechol to conditions sufficient to convert catechol to muconic acid (e.g., under oxidative conditions); and (d) subjecting muconic acid to conditions sufficient to convert muconic acid to adipic acid. In some embodiments, the method comprises subjecting muconic acid to reductive conditions.

In some embodiments, lignin is converted to vanillic acid by any method disclosed herein. In some embodiments, vanillic acid is converted to 2-methoxyphenol by any method disclosed herein. In some embodiments, 2-methoxyphenol is converted to catechol by any method disclosed herein. In some embodiments, catechol is converted to muconic acid by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of Muconic Acid to Adipic Acid

In some embodiments, muconic acid is separated or extracted from any residual material (e.g., lignin, vanillic acid, 2-methoxyphenol, and/or catechol) prior to converting it to adipic acid, and in other embodiments, is not separated or extracted from any residual material prior to converting it to adipic acid.

In some embodiments, a carbon-carbon unsaturated group (e.g., an alkenyl group) on muconic acid is reduced. In some embodiments, muconic acid is catalytically hydrogenated. In some embodiments, muconic acid is contacted with hydrogen and (e.g., hydrogen gas) and a metal catalyst. In some embodiments, the metal catalyst is platinum, palladium (e.g., Pd/C), rhodium, ruthenium, Raney nickel, and Urushibara nickel.

In some embodiments, the reduction occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

ii. Isolation of Adipic Acid

In some embodiments, the muconic acid is isolated from any remaining lignin, vanillic acid, 2-methoxyphenol, catechol, methanol, and/or muconic acid. In some embodiments, adipic acid is isolated by any suitable method. In some embodiments, adipic acid is isolated from any remaining lignin, vanillic acid, 2-methoxyphenol, catechol, and/or muconic acid by use of a solvent. In some embodiments, the adipic acid is isolated from any remaining lignin, vanillic acid, 2-methoxyphenol, catechol, methanol, and/or muconic acid by use of at least two solvent.

iii. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### F. Acrylates and Butadiene

Disclosed herein, in certain embodiments, is acrylates and butadiene obtained from lignin. Further disclosed herein, in certain embodiments, is a method of obtaining acrylates and butadiene from lignin. It is to be understood that provided in various embodiments herein are both (1) acrylates and butadiene prepared according to any of the processes described herein; and (2) any method described herein for obtaining acrylates and butadiene from a lignin source.

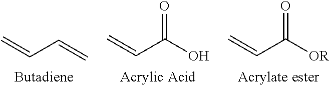

Disclosed herein, in certain embodiments, is a method of obtaining acrylates and butadiene from lignin, comprising: (a) subjecting lignin to oxidative conditions sufficient to produce vanillic acid, (b) subjecting vanillic acid to conditions sufficient to convert vanillic acid to catechol; (c) subjecting catechol to conditions sufficient to convert catechol to hexa-2,4-dienedioic acid; and (d) subjecting hexa-2,4-dienedioic acid to conditions (e.g., olefin metathesis) sufficient to convert hexa-2,4-dienedioic acid to acrylic acid and butadiene. In some embodiments, the method comprises esterifying hexa-2,4-dienedioic acid before subjecting it to olefin metathesis. In specific embodiments, olefin metathesis is performed by adding an additional olefin, e.g., ethylene, and is catalyzed, e.g., by the use of a metal, such as nickel, tungsten, ruthenium, molybdenum, or the like, or combinations thereof. In some embodiments, the olefin metathesis catalyst is a black box catalyst (e.g., WCl₆/SnMe₄ or Re₂O₇Al₂O₃); a titanocene-based catalyst (3.g., Cp₂Ti(u-Cl)(u-CH₂)AlMe₂); a Schrock W, Mo, or Re catalyst, including an arylimido complex of Mo (e.g., (Ar′N)(RO)₂Mo═CHR′, wherein Ar′ is aryl, e.g., 2,6-diisopropylphenyl, and R′ is any suitable substituent, e.g., alkyl or aryl, and R is neopentyl or neophyl (CMe₂Ph)); or a Grubbs Ru catalyst.

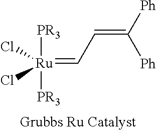

Disclosed herein, in certain embodiments, is a method of obtaining acrylates and butadiene from lignin, comprising: (a) subjecting lignin to oxidative conditions sufficient to produce vanillic acid, (b) subjecting vanillic acid to conditions sufficient to convert vanillic acid to catechol; (c) subjecting catechol to conditions sufficient to convert catechol to hexa-2,4-dienedioic acid; (d) esterifying hexa-2,4-dienedioic acid and (e) subjecting the ester to conditions sufficient to convert the ester to acrylate ester and butadiene.

In some embodiments, lignin is converted to vanillic acid by any method disclosed herein. In some embodiments, vanillic acid is converted to catechol by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of hexa-2,4-dienedioic acid to Butadiene and an Acrylate

In some embodiments, catechol is separated or extracted from any residual material (e.g., lignin, vanillic acid, and/or 2-methoxyphenol) prior to converting it to a downstream compound, e.g., hexa-2,4-dienedioic acid. In some embodiments, catechol is contacted with a catechol dioxygenase (e.g., a 1,2-catechol dioxygenase) under conditions sufficient to yield hexa-2,4-dienedioic acid.

In some embodiments, hexa-2,4-dienedioic acid is separated or extracted from any residual material (e.g., lignin, vanillic acid, 2-methoxyphenol, methanol, and/or catechol) prior to converting it to butadiene and acrylate, and in other embodiments, is not separated or extracted from any residual material prior to converting it to butadiene and acrylate. In some embodiments, hexa-2,4-dienedioic acid is subjected to olefin metathesis under conditions sufficient to yield butadiene and acrylic acid. In some embodiments, hexa-2,4-dienedioic acid is esterified under conditions sufficient to yield the ester. In some embodiments, the ester is subjected to olefin metathesis under conditions sufficient to yield butadiene and acrylate ester.

In some embodiments, the reaction occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

ii. Isolation of Butadiene

In some embodiments, the butadiene is isolated from any remaining lignin, vanillic acid, catechol, hexa-2,4-dienedioic acid (or and ester thereof), and/or acrylate. In some embodiments, butadiene is isolated by any suitable method. In some embodiments, butadiene is isolated from any remaining lignin, methanol, vanillic acid, catechol, hexa-2,4-dienedioic acid (or and ester thereof), and/or acrylate by use of a solvent. In some embodiments, the butadiene is isolated from any remaining lignin, vanillic acid, methanol, catechol, hexa-2,4-dienedioic acid (or and ester thereof), and/or acrylate by use of at least two solvents.

iii. Isolation of an Acrylate

In some embodiments, the acrylate is isolated from any remaining lignin, vanillic acid, catechol, hexa-2,4-dienedioic acid (or an ester thereof) and/or butadiene. In some embodiments, an acrylate is isolated by any suitable method. In some embodiments, an acrylate is isolated from any remaining lignin, vanillic acid, catechol, methanol, hexa-2,4-dienedioic acid (or an ester thereof), and/or butadiene by use of a solvent. In some embodiments, an acrylate is isolated from any remaining lignin, vanillic acid, methanol, catechol, hexa-2,4-dienedioic acid (or an ester thereof), and/or butadiene by use of at least two solvent.

iv. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### G. 2-Aminophenol

Disclosed herein, in certain embodiments, is 2-aminophenol (2-AP) obtained from lignin. In some embodiments, the 2-AP is suitable for use in azo dyes, photographic developer, paint corrosion inhibitor, and as a monomer in benzoxazole polymers. Further disclosed herein, in certain embodiments, is a method of obtaining 2-aminophenol from lignin. It is to be understood that provided in various embodiments herein are both (1) 2-aminophenol prepared according to any of the processes described herein; and (2) any method described herein for obtaining 2-aminophenol from a lignin source.

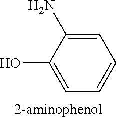

Disclosed herein, in certain embodiments, is a method of obtaining 2-aminophenol from lignin, comprising: (a) subjecting lignin to oxidative conditions sufficient to produce vanillic acid, (b) subjecting vanillic acid to conditions sufficient to convert vanillic acid to 4-amino-3-methoxybenzoic acid; and (c) subjecting 4-amino-3-methoxybenzoic acid to conditions sufficient to convert 4-amino-3-methoxybenzoic acid to 2-aminophenol.

In some embodiments, lignin is converted to vanillic acid by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of 4-amino-3-methoxybenzoic acid to 2-aminophenol—Pathway 1

In some embodiments, vanillic acid is separated or extracted from any residual material (e.g., lignin) prior to converting it to 4-amino-3-methoxybenzoic acid, and in other embodiments, is not separated or extracted from any residual material prior to converting it to 4-amino-3-methoxybenzoic acid.

In some embodiments, vanillic acid is converted to 4-amino-3-methoxybenzoic acid by amination. In some embodiments, vanillic acid is contacted with ammonia (NH₃) and a metal catalyst and/or a zeolite catalyst. In some embodiments, vanillic acid is contacted with ammonia (NH₃) and acid washed H-151 alumina or another catalyst as described in U.S. Pat. No. 5,214,210, which is incorporated herein for such disclosure. In some embodiments, the contacting occurs under conditions sufficient to convert vanillic acid to 4-amino-3-methoxybenzoic acid.

In some embodiments, 4-amino-3-methoxybenzoic acid is separated or extracted from any residual material (e.g., lignin and/or vanillic acid) prior to converting it to 2-aminophenol, and in other embodiments, is not separated or extracted from any residual material prior to converting it to 2-aminophenol.

In some embodiments, 4-amino-3-methoxybenzoic acid is converted to 2-aminophenol. In some embodiments, converting 4-amino-3-methoxybenzoic acid to 2-aminophenol comprises (a) demethylating 4-amino-3-methoxybenzoic acid; and (b) decarboxylating 4-amino-3-methoxybenzoic acid such that 2-methoxyphenol is produced. In some embodiments, decarboxylation and demethylation comprise contacting the 4-amino-3-methoxybenzoic acid with supercritical or near critical water. As used herein, “supercritical water” is water heated to about 400° C. and at about 23 MPa. As used herein, “near critical water” is water that has a temperature and/or pressure about 1% less than, about 2% less than, about 3% less than, about 5% less than, about 10% less than, or about 15% less than is necessary for the water to become supercritical.

In some embodiments, decarboxylating 4-amino-3-methoxybenzoic acid comprises contacting 4-amino-3-methoxybenzoic acid with a decarboxylase under conditions that instigate or maximize the enzymatic reaction. In some decarboxylating 4-amino-3-methoxybenzoic acid comprises contacting 4-amino-3-methoxybenzoic acid with a decarboxylase and heating the combination. In some embodiments, the decarboxylase/4-amino-3-methoxybenzoic acid combination is heated to about 50° C., about 100° C., about 150° C., about 200° C., about 250° C., about 300° C., about 350° C., or about 350° C.

In some embodiments, the demethylation and decarboxylation occur simultaneously. In some embodiments, the demethylation occurs before the decarboxylation. In some embodiments, the demethylation product is isolated before decarboxylation. In some embodiments, the decarboxylation occurs before the demethylation. In some embodiments, the decarboxylation product is isolated before demethylation.

In some embodiments, the reaction occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

In some embodiments, methanol is generated from the demethylation of 4-amino-3-methoxybenzoic acid. In some embodiments, methanol is isolated from the reaction mixture.

In some embodiments, 2-methoxyaniline is generated from the decarboxylation of 4-amino-3-methoxybenzoic acid. In some embodiments, 2-methoxyaniline is isolated from the reaction mixture.

ii. Conversion of 4-amino-3-methoxybenzoic acid to 2-aminophenol—Pathway 2

In some embodiments, vanillic acid is separated or extracted from any residual material (e.g., lignin) prior to converting it to 4-amino-3-methoxybenzoic acid, and in other embodiments, is not separated or extracted from any residual material prior to converting it to 4-amino-3-methoxybenzoic acid.

In some embodiments, vanillic acid is converted to catechol by 4-demethylation and decarboxylation (e.g., following contact with super critical or near critical water). In some embodiments, the demethylation of catechol generates methanol. In some embodiments, the methanol is isolated.

In some embodiments, catechol is contacted with ammonia (NH₃) and a metal catalyst and/or a zeolite catalyst. In some embodiments, catechol is contacted with ammonia (NH₃) and acid washed H-151 alumina or another catalyst as described in U.S. Pat. No. 5,214,210. In some embodiments, the contacting occurs under conditions sufficient to convert catechol to 2-aminophenol.

iii. Isolation of 2-aminophenol

In some embodiments, the 2-aminophenol is isolated from any remaining lignin, vanillic acid, and/or 4-amino-3-methoxybenzoic acid. In some embodiments, 2-aminophenol is isolated by any suitable method. In some embodiments, 4-amino-3-methoxybenzoic acid is isolated from any remaining lignin, vanillic acid, and/or 4-amino-3-methoxybenzoic acid by use of a solvent. In some embodiments, the 4-amino-3-methoxybenzoic acid is isolated from any remaining lignin, vanillic acid, and/or 4-amino-3-methoxybenzoic acid by use of at least two solvents.

iv. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### H. Aniline

Disclosed herein, in certain embodiments, is aniline obtained from lignin. In some embodiments, the aniline is suitable for use as MDI starting material, in dyes and pigments, in the generation of rubber, and in agrochemicals. Further disclosed herein, in certain embodiments, is a method of obtaining aniline from lignin. It is to be understood that provided in various embodiments herein are both (1) aniline prepared according to any of the processes described herein; and (2) any method described herein for obtaining aniline from a lignin source.

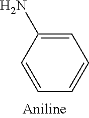

Disclosed herein, in certain embodiments, is a method of obtaining aniline from lignin, comprising: (a) subjecting lignin to oxidative conditions sufficient to produce vanillic acid, (b) subjecting vanillic acid to conditions sufficient to convert vanillic acid to 4-amino-3-methoxybenzoic acid; (c) subjecting 4-amino-3-methoxybenzoic acid to conditions sufficient to convert 4-amino-3-methoxybenzoic acid to 2-aminophenol; and (d) subjecting 2-aminophenol to conditions sufficient to convert 2-aminophenol to aniline.

In some embodiments, lignin is converted to vanillic acid by any method disclosed herein. In some embodiments, vanillic acid is converted to 2-aminophenol by any method disclosed herein.

In some embodiments, the reaction occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

i. Conversion of 2-aminophenol to aniline

In some embodiments, 2-aminophenol is separated or extracted from any residual material (e.g., lignin, vanillic acid, methanol, and/or 4-amino-3-methoxybenzoic) prior to converting it to aniline, and in other embodiments, is not separated or extracted from any residual material prior to converting it to aniline.

In some embodiments, methanol is generated from the demethylation of 4-amino-3-methoxybenzoic acid. In some embodiments, methanol is isolated from the reaction mixture.

ii. Isolation of aniline

In some embodiments, the aniline is isolated from any remaining lignin, vanillic acid, 4-amino-3-methoxybenzoic acid, methanol, and/or 2-aminophenol. In some embodiments, aniline is isolated by any suitable method. In some embodiments, aniline is isolated from any remaining lignin, vanillic acid, methanol, 4-amino-3-methoxybenzoic acid, and/or 2-amiophenol by use of a solvent. In some embodiments, the aniline is isolated from any remaining lignin, vanillic acid, methanol, 4-amino-3-methoxybenzoic acid, and/or 2-aminophenol by use of at least two solvents.

iii. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

I. o-Phenylenediamine

Disclosed herein, in certain embodiments, is o-Phenylenediamine (OPDA) obtained from lignin. In some embodiments, the OPDA obtained from lignin is suitable for use in Azo dyes, as a polymer monomer, or as an anti-oxidant. Further disclosed herein, in certain embodiments, is a method of obtaining o-Phenylenediamine from lignin. It is to be understood that provided in various embodiments herein are both (1) o-Phenylenediamine prepared according to any of the processes described herein; and (2) any method described herein for obtaining o-Phenylenediamine from a lignin source. A particular benefit of the following process is it allows the generation of OPDA with the use of nitric acid.

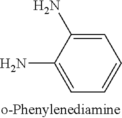

Disclosed herein, in certain embodiments, is a method of obtaining OPDA from lignin, comprising: (a) subjecting lignin to oxidative conditions sufficient to produce vanillic acid, (b) subjecting vanillic acid to conditions sufficient to convert vanillic acid to 4-amino-3-methoxybenzoic acid; and (c) subjecting 4-amino-3-methoxybenzoic acid to conditions sufficient to convert 4-amino-3-methoxybenzoic acid to OPDA.

In some embodiments, lignin is converted to vanillic acid by any method disclosed herein.

In some embodiments, the lignin is isolated from biomass (e.g., lignocellulose biomass) by any method disclosed herein. In some embodiments, the lignin is not isolated from biomass. In some embodiments, the contacting occurs under heating, pressurizing and/or agitating (e.g., stirring or mixing). In some embodiments, heating comprises heating to at least room temperature, at least 25° C., at least 35° C., at least 50° C., at least 70° C., at least 80° C., at least 90° C., or at least 100° C.

i. Conversion of 4-amino-3-methoxybenzoic acid to OPDA

In some embodiments, vanillic acid is separated or extracted from any residual material (e.g., lignin) prior to converting it to 4-amino-3-methoxybenzoic acid, and in other embodiments, is not separated or extracted from any residual material prior to converting it to 4-amino-3-methoxybenzoic acid.

In some embodiments, vanillic acid is converted to 4-amino-3-methoxybenzoic acid by amination. In some embodiments, vanillic acid is contacted with ammonia (NH₃) and a metal catalyst or a zeolite catalyst. In some embodiments, vanillic acid is contacted with ammonia (NH₃) and acid washed H-151 alumina or another catalyst as described in U.S. Pat. No. 5,214,210. In some embodiments, the contacting occurs under conditions sufficient to convert vanillic acid to 4-amino-3-methoxybenzoic acid.

In some embodiments, 4-amino-3-methoxybenzoic acid is separated or extracted from any residual material (e.g., lignin and/or vanillic acid) prior to converting it to OPDA, and in other embodiments, is not separated or extracted from any residual material prior to converting it to OPDA.

In some embodiments, 4-amino-3-methoxybenzoic acid is converted to OPDA. In some embodiments, converting 4-amino-3-methoxybenzoic acid to OPDA comprises decarboxylating 4-amino-3-methoxybenzoic acid such that OPDA is produced. In some embodiments, decarboxylation comprises contacting the 4-amino-3-methoxybenzoic acid with supercritical or near critical water. As used herein, “supercritical water” is water heated to about 400° C. and at about 23 MPa. As used herein, “near critical water” is water that has a temperature and/or pressure about 1% less than, about 2% less than, about 3% less than, about 5% less than, about 10% less than, or about 15% less than is necessary for the water to become supercritical.

In some embodiments, decarboxylating 4-amino-3-methoxybenzoic acid comprises contacting 4-amino-3-methoxybenzoic acid with a decarboxylase under conditions that instigate or maximize the enzymatic reaction. In some embodiments, decarboxylating 4-amino-3-methoxybenzoic acid comprises contacting 4-amino-3-methoxybenzoic acid with a decarboxylase and/or heating the combination. In some embodiments, the decarboxylase/4-amino-3-methoxybenzoic acid combination or 4-amino-3-methoxybenzoic acid is heated to about 50° C., about 100° C., about 150° C., about 200° C., about 250° C., about 300° C., about 350° C., or about 350° C.

In some embodiments, the reaction occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

In some embodiments, methanol is generated from the demethylation of 4-amino-3-methoxybenzoic acid. In some embodiments, methanol is isolated from the reaction mixture.

ii. Isolation of o-Phenylenediamine

In some embodiments, the o-Phenylenediamine is isolated from any remaining lignin, vanillic acid, methanol, and/or 4-amino-3-methoxybenzoic acid. In some embodiments, o-Phenylenediamine is isolated by any suitable method. In some embodiments, o-Phenylenediamine is isolated from any remaining lignin, vanillic acid, methanol, and/or 4-amino-3-methoxybenzoic acid by use of a solvent. In some embodiments, the aniline is isolated from any remaining lignin, vanillic acid, methanol, and/or 4-amino-3-methoxybenzoic acid by use of at least two solvents.

iii. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

J. Caprolactam

Disclosed herein, in certain embodiments, is caprolactam obtained from lignin. Further disclosed herein, in certain embodiments, is a method of obtaining caprolactam from lignin. It is to be understood that provided in various embodiments herein are both (1) caprolactam prepared according to any of the processes described herein; and (2) any method described herein for obtaining caprolactam from a lignin source.

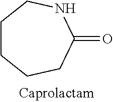

Disclosed herein, in certain embodiments, is a method of obtaining caprolactam from lignin, comprising: (a) subjecting lignin to oxidative conditions sufficient to produce vanillic acid, (b) subjecting vanillic acid to conditions sufficient to convert vanillic acid to cyclohexanecarboxylic acid; and (d) subjecting cyclohexanecarboxylic acid to conditions sufficient to convert cyclohexanecarboxylic acid to caprolactam.

In some embodiments, lignin is converted to vanillic acid by any method disclosed herein.

In some embodiments, the reaction occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

i. Conversion of Cyclohexanecarboxylic Acid into Caprolactam

In some embodiments, vanillic acid is separated or extracted from any residual material (e.g., lignin) prior to converting it to cyclohexanecarboxylic acid, and in other embodiments, is not separated or extracted from any residual material prior to converting it to cyclohexanecarboxylic acid. In some embodiments, vanillic acid is converted to cyclohexanecarboxylic acid under reducing conditions.

In some embodiments, a carbon-carbon unsaturated group (e.g., an alkenyl group) on vanillic acid is reduced (e.g., so as to produce a cyclohexanecarboxylic acid). In some embodiments, vanillic acid is catalytically hydrogenated. In some embodiments, vanillic acid is contacted with hydrogen (e.g., hydrogen gas) and a metal catalyst. In some embodiments, the metal catalyst is platinum, palladium (e.g., Pd/C), rhodium, ruthenium, Raney nickel, and Urushibara nickel.

In some embodiments, the reduction occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

In some embodiments, cyclohexanecarboxylic acid is separated or extracted from any residual material (e.g., lignin, or vanillic acid) prior to converting it to caprolactam, and in other embodiments, is not separated or extracted from any residual material prior to converting it to caprolactam.

In some embodiments, cyclohexanecarboxylic acid is converted to caprolactam by contacting cyclohexanecarboxylic acid with nitrosylsulfuric acid (NO(HSO₄)). In some embodiments, nitrosulfuric acid is generated from ammonia, oxygen, and oleum. In some embodiments, the contacting occurs under conditions that instigate or maximize the reaction (e.g., heating or high pressure). In some embodiments, the contacting occurs under heating, pressurizing and/or agitating (e.g., stirring or mixing). In some embodiments, the reaction mixture is heated to at least room temperature, at least 25° C., at least 35° C., at least 50° C., at least 70° C., at least 80° C., at least 90° C., or at least 100° C. In some embodiments, the reaction mixture is pressurized to at least 20 psi, at least 30 psi, at least 40 psi, 50 psi, at least 100 psi, at least 200 psi, at least 300 psi, at least 500 psi, at least 1000 psi or the like.

In some embodiments, methanol is generated from the reduction of cyclohexanecarboxylic acid. In some embodiments, methanol is isolated from the reaction mixture.

ii. Isolation of Caprolactam

In some embodiments, the caprolactam is isolated from any remaining lignin, vanillic acid, methanol, and/or cyclohexanecarboxylic acid. In some embodiments, caprolactam is isolated by any suitable method. In some embodiments, caprolactam is isolated from any remaining lignin, vanillic acid, methanol, and/or cyclohexanecarboxylic acid by use of a solvent. In some embodiments, the aniline is isolated from any remaining lignin, vanillic acid, methanol, and/or cyclohexanecarboxylic acid by use of at least two solvents.

iii. Use of a Reactor

In some embodiments, the biomass is contacted with an enzyme in a reactor. In some embodiments, the lignin is separated from the biomass in a reactor. In some embodiments, the reactor is, by way of non-limiting example, a flow reactor (e.g., a batch-flow reactor and a continuous flow reactor). In some embodiments, the reactor is a large scale industrial reactor.

### EXAMPLES

### Example 1

### Generation of Vanillic Acid

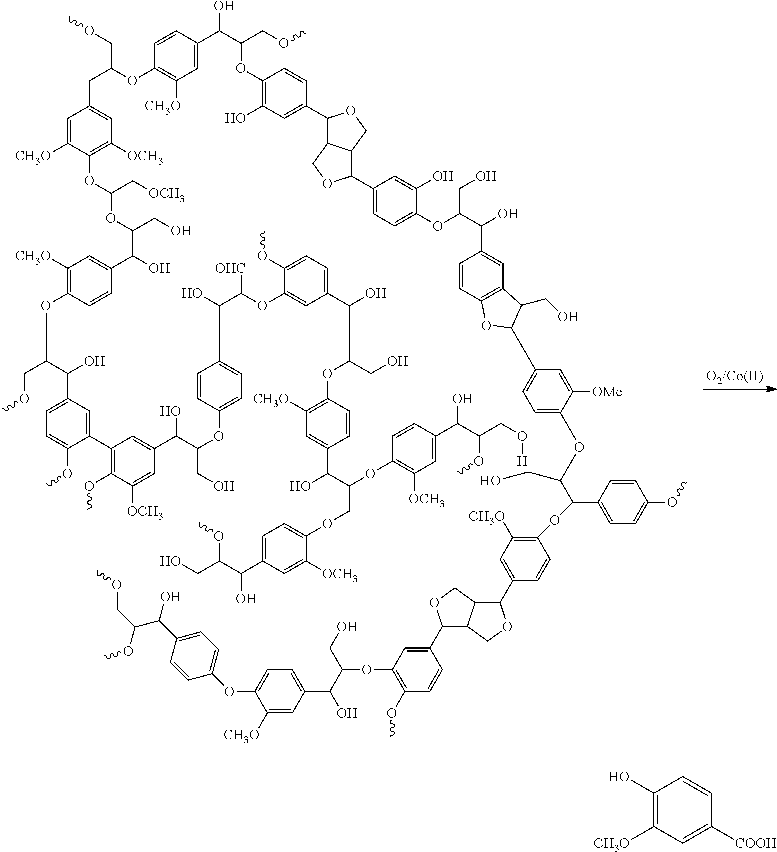

Lignin is oxidized to vanillic acid by contacting lignin with molecular oxygen and a cobalt (II) metal catalyst. In alternative instances, the metal catalyst MeReO₃ can be used.

### Example 2

### Generation of 2-Methoxyphenol (aka Guaiacol)

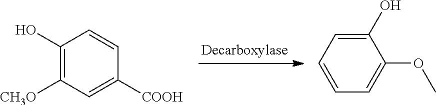

Lignin is converted into 2-methoxyphenol by first contacting lignin with molecular oxygen and a cobalt (II) catalyst to generate vanillic acid. Next, the vanillic acid is isolated from the lignin. Then, the vanillic acid is decarboxylated with a decarboxylase

### Example 3

### Generation of Catechol

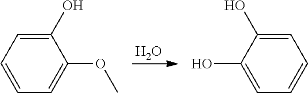

Lignin is converted into catechol by first contacting lignin with molecular oxygen and a cobalt (II) catalyst to generate vanillic acid. Next, the vanillic acid is isolated from the lignin. Then, the vanillic acid is decarboxylated with a decarboxylase, generating 2-methoxyphenol. Finally, 2-methoxyphenol is contacted with super critical water to generate catechol.

### Example 4

### Generation of Phenol

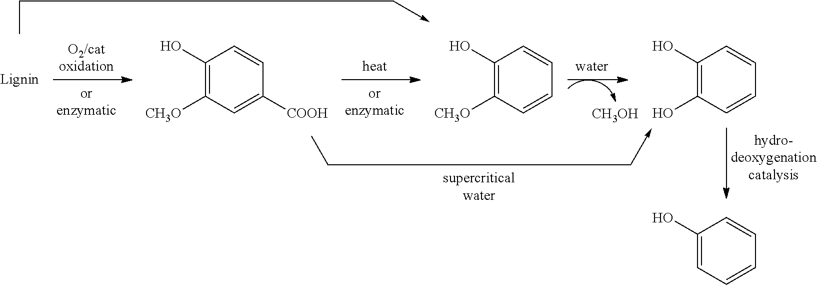

Lignin is converted into catechol by first contacting lignin with molecular oxygen and a cobalt (II) catalyst to generate vanillic acid. Next, the vanillic acid is isolated from the lignin. Then, the vanillic acid is decarboxylated and demethylated by contacting it with super critical water to generate catechol. Finally, the catechol is contacted with hydrogen and catalyst (e.g., BiVO₄, CuWO₄, Cu_{0.75}Zn_{0.25}WO₄, or CuWO₄+Ce, CuO—Al₂O₃) to generate phenol.

### Example 5

### Generation of Muconic Acid

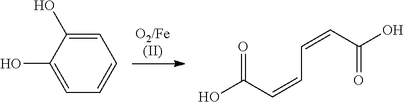

Lignin is converted into muconic acid by first contacting lignin with molecular oxygen and a cobalt (II) catalyst to generate vanillic acid. Next, the vanillic acid is isolated from the lignin. Then, the vanillic acid is decarboxylated with a decarboxylase, generating 2-methoxyphenol. Next, 2-methoxyphenol is contacted with super critical water to generate catechol. Finally, the catechol is contacted with molecular oxygen and Fe(II) to generate muconic acid.

### Example 6

### Generation of Adipic Acid

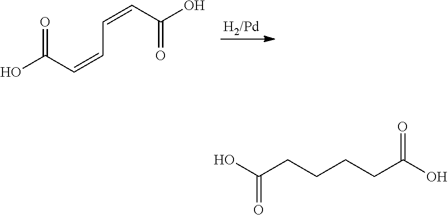

Lignin is converted into muconic acid by first contacting lignin with molecular oxygen and a cobalt (II) catalyst to generate vanillic acid. Next, the vanillic acid is isolated from the lignin. Then, the vanillic acid is decarboxylated with a decarboxylase, generating 2-methoxyphenol. Next, 2-methoxyphenol is contacted with super critical water to generate catechol. Then, the catechol is contacted with molecular oxygen and Fe(II) to generate muconic acid. Finally, muconic acid is subjected to reducing conditions via exposure to H₂ and Pd.

### Example 7

### Generation of Butadiene and Acrylic Acid

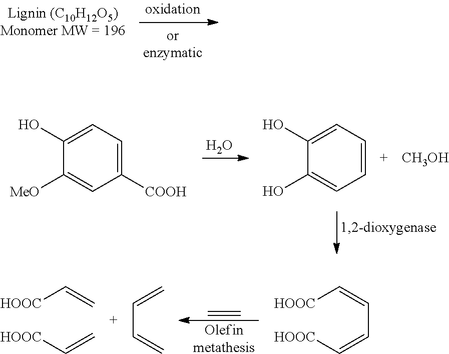

Lignin is converted into muconic acid by first contacting lignin with molecular oxygen and a cobalt (II) catalyst to generate vanillic acid. Next, the vanillic acid is isolated from the lignin. Then, the vanillic acid is decarboxylated with a decarboxylase, generating 2-methoxyphenol. Next, 2-methoxyphenol is contacted with super critical water to generate catechol. Then, the catechol is contacted with catechol 1,2-dioxygenase to generate hexa-2,4-dienedioic acid. Finally, hexa-2,4-dienedioic acid is subjected to catalytic olefin metathesis to generate butadiene and acrylic acid.

### Example 8

### Generation of 2-aminophenol

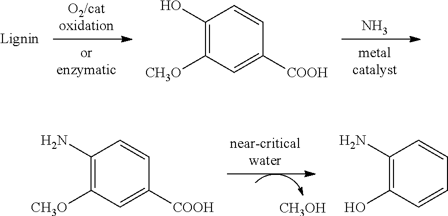

Lignin is converted into 2-aminophenol by first contacting lignin with molecular oxygen and a cobalt (II) catalyst to generate vanillic acid. Next, the vanillic acid is isolated from the lignin. Then, the vanillic acid is aminated by contacting it with ammonia and acid washed H-151 alumina or another catalyst as described in U.S. Pat. No. 5,214,210. Next, 4-amino-3-methoxybenzoic acid is contacted with near-critical water, generating 2-aminophenol.

### Example 9

### Generation of Aniline

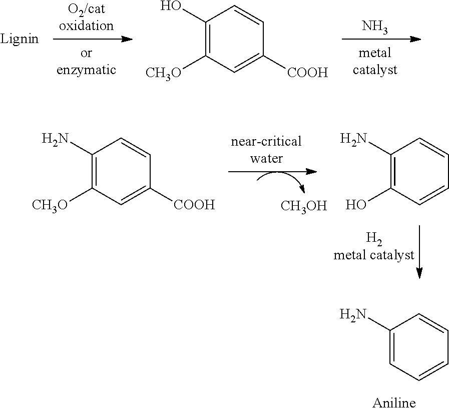

Lignin is converted into aniline by first contacting lignin with molecular oxygen and a cobalt (II) catalyst to generate vanillic acid. Next, the vanillic acid is isolated from the lignin. Then, the vanillic acid is aminated by contacting it with ammonia and Pd or Nb₂O₅. Next, 4-amino-3-methoxybenzoic acid is contacted with near-critical water, generating 2-aminophenol. Finally, 2-aminophenol is reduced by contact with H₂ and Pd.

### Example 10

### Generation of OPDA

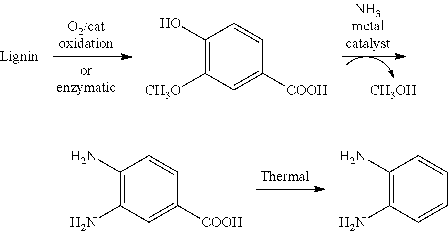

Lignin is converted into OPDA by first contacting lignin with molecular oxygen and a cobalt (II) catalyst to generate vanillic acid. Next, the vanillic acid is isolated from the lignin. Then, the vanillic acid is aminated by contacting it with ammonia and acid washed H-151 alumina or another catalyst as described in U.S. Pat. No. 5,214,210. Next, 4-amino-3-methoxybenzoic acid is contacted with super critical water to produce OPDA.

### Example 11

### Generation of Caprolactam

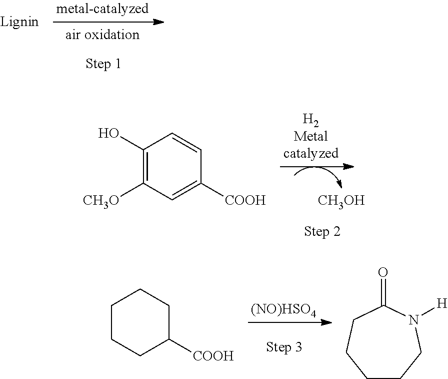

Lignin is converted into caprolactam by first contacting lignin with molecular oxygen and a cobalt (II) catalyst to generate vanillic acid. Next, the vanillic acid is isolated from the lignin. Then, the vanillic acid is reduced to cyclohexanecarboxylic acid by contacting it with hydrogen and Pd. Next, cyclohexanecarboxylic acid is contacted with nitrosylsulfuric acid, generating caprolactam.

1. A method of obtaining vanillic acid from lignin, comprising: subjecting lignin to oxidative conditions sufficient to produce vanillic acid.

2. The method of claim 1, wherein the lignin is isolated from biomass.

3. The method of claim 1, comprising contacting lignin with a ligninase.

4. The method of claim 1, comprising contacting lignin with an oxidase, a peroxidase, or both.

5. (canceled)

6. The method of claim 1, comprising contacting lignin with molecular oxygen, hydrogen peroxide, or both.

7. The method of claim 1, comprising contacting lignin with (a) an oxidizing agent;
and (b) a metal catalyst.

8. The method of claim 7, wherein the metal catalyst comprises Co, Rh, Ir, MeReO₃, or combinations thereof.

9-24. (canceled)

25. The method of claim 1 further comprising:
a. obtaining 2-methoxyphenol by subjecting the vanillic acid to conditions sufficient to produce 2-methoxyphenol; and/or
b. obtaining catechol by (i) subjecting the vanillic acid to conditions sufficient to produce 2-methoxyphenol; and (ii) subjecting 2-methoxyphenol to conditions sufficient to produce catechol;

26. The method of claim 1 further comprising: obtaining phenol by (i) subjecting the vanillic acid to conditions sufficient to produce 2-methoxyphenol; (ii) subjecting 2-methoxyphenol to conditions sufficient to produce catechol; and (iii) subjecting catechol to conditions sufficient to produce phenol;

27. The method of claim 1 further comprising:
a. obtaining acrylic acid and butadiene by (i) subjecting the vanillic acid to conditions sufficient to produce 2-methoxyphenol; (ii) subjecting 2-methoxyphenol to conditions sufficient to produce catechol; (iii) subjecting catechol to conditions sufficient to convert catechol to hexa-2,4-dienedioic acid; and (iv) subjecting hexa-2,4-dienedioic acid to conditions sufficient to convert hexa-2,4-dienedioic acid to acrylic acid and butadiene; and/or
b. obtaining an acrylate ester and butadiene by (i) subjecting the vanillic acid to conditions sufficient to produce 2-methoxyphenol; (ii) subjecting 2-methoxyphenol to conditions sufficient to produce catechol; (iii) subjecting catechol to conditions sufficient to convert catechol to hexa-2,4-dienedioic acid; (iv) esterifying hexa-2,4-dienedioic acid;
and (v) subjecting the ester of hexa-2,4-dienedioic acid to conditions sufficient to convert the ester to an acrylate ester and butadiene.

28. A method of obtaining muconic acid comprising:
a. subjecting lignin to oxidative conditions sufficient to produce vanillic acid; and
b. subjecting the vanillic acid to conditions sufficient to produce muconic acid.

29. A method of obtaining adipic acid comprising:
a. subjecting lignin to oxidative conditions sufficient to produce vanillic acid;
b. subjecting the vanillic acid to conditions sufficient to produce muconic acid; and
c. subjecting muconic acid to conditions sufficient to produce adipic acid.

30. The method of claim 1 further comprising: obtaining 2-aminophenol by (i) subjecting the vanillic acid to conditions sufficient to convert vanillic acid to 4-amino-3-methoxybenzoic acid; and (c) subjecting 4-amino-3-methoxybenzoic acid to conditions sufficient to convert 4-amino-3-methoxybenzoic acid to 2-aminophenol;

31. The method of claim 1 further comprising: obtaining aniline by (i) subjecting the vanillic acid to conditions sufficient to convert vanillic acid to 4-amino-3-methoxybenzoic acid; (ii) subjecting 4-amino-3-methoxybenzoic acid to conditions sufficient to convert 4-amino-3-methoxybenzoic acid to 2-aminophenol; and (iii) a subjecting 2-aminophenol to conditions sufficient to produce aniline; and/or

32. The method of claim 1 further comprising: obtaining o-phenylenediamine by (i) subjecting vanillic acid to conditions sufficient to convert vanillic acid to 4-amino-3-methoxybenzoic acid; and (ii) subjecting 4-amino-3-methoxybenzoic acid to conditions sufficient to convert 4-amino-3-methoxybenzoic acid to o-phenylenediamine.

33. The method of claim 1 further comprising obtaining caprolactam by (i) subjecting vanillic acid to conditions sufficient to convert vanillic acid to cyclohexanecarboxylic acid; and (ii) subjecting cyclohexanecarboxylic acid to conditions sufficient to convert cyclohexanecarboxylic acid to caprolactam.

34. The method of claim 1 wherein subjecting lignin to oxidative conditions sufficient to produce vanillic acid comprises (1) contacting lignin with a catalytic amount of nitroarene catalyst to oxidize lignin and provide an inactive nitroarene byproduct; and (2) recycling the nitroarene byproduct to regenerate the nitroarene catalyst.

35. The method of claim 34, wherein recycling the nitroarene byproduct to regenerate the nitroarene catalyst comprises contacting the nitroarene byproduct with molecular oxygen and a metal catalyst.

36. The method of claim 34, wherein recycling the nitroarene byproduct to regenerate the nitroarene catalyst comprises contacting the nitroarene byproduct with molecular oxygen and an N-oxygenase.

37. The method of claim 36, wherein the N-oxygenase is AurF from Streptomyces thioluteus.
